(19) United States
(12) Patent Application Publication
(10) Pub. No.: US 2024/0408599 A1
(43) Pub. Date: Dec. 12, 2024

(54) DEVICE AND METHOD FOR BACTERIA DETECTION

(71) Applicant: TECHNION RESEARCH & DEVELOPMENT FOUNDATION LIMITED, Haifa (IL)

(72) Inventors: Gilad YOSSIFON, Haifa (IL); Sinwook PARK, Haifa (IL); Barak SABBAGH, Haifa (IL); Yechezkel KASHI, Haifa (IL); Shay KIRZNER, Haifa (IL); Keren ROSENBLAU-BUHNIK, Haifa (IL)

(21) Appl. No.: 18/702,312

(22) PCT Filed: Oct. 27, 2022
(86) PCT No.: PCT/IL2022/051144

§ 371 Date: Apr. 17, 2024

Related U.S. Application Data
(60) Provisional application No. 63/272,383, filed on Oct. 27, 2021.

(51) Int. Cl. B01L 3/00; C12Q 1/24; C12Q 1/6888

(52) U.S. Cl. CPC B01L 3/502761; B01L 3/502715; C12Q 1/24; C12Q 1/6888; B01L 2300/0645; B01L 2400/0424

(57) ABSTRACT

The present invention provides, in some embodiments, a device, kit and method, for detection of a microorganism in a food sample.

### CROSS REFERENCE TO RELATED APPLICATIONS

This application claims the benefit of priority to U.S. Provisional Patent Application No. 63/272,383 filed Oct. 27, 2021, entitled “DEVICE AND METHOD FOR BACTERIA DETECTION” the contents of which are incorporated herein by reference in their entirety.

### FIELD OF INVENTION

The present invention relates to a device and a method for pathogen detection as well as bacteria in general.

### BACKGROUND

Millions of people are annually affected by food illnesses, with increasing death cases. Food contaminations are mostly monitored in plants using time consuming methods with analysis time of up to a week. By that time, some of the contaminated products are released to the market and consumed. This food testing paradox is demonstrated by the enormous rate of 50 weekly recalls across the EU and USA.

Nowadays, pathogen detection mainly relies on culture-based methods, considered as the gold standard as being selective, sensitive and accurate, yet those methods suffer from the major drawback of slow analysis time of up to one week. Immunological methods are more rapid, yet still do not provide real time results and are not sensitive enough. PCR-based assays, based on amplification of the pathogen specific sequences, are becoming very popular detection methods mainly due to their sensitivity and short analysis time. Still, their sensitivity is not high enough, and are unreliable below a thousand cells per sample. Therefore, these methods require an additional long (>10 hours) culture-enrichment step.

There is a great need for development of a highly sensitive, affordable, easy-to-handle, portable and rapid detection device, resulting in a decrease in food contaminations.

Dielectrophoresis (DEP) is an induced motion of a particle that is caused by a non-uniform electric field acting on the dipole it induces in the particle. The particles are either attracted (positive DEP (pDEP)) to high field gradients or repelled (negative DEP (nDEP)) from them. Dielectrophoretic forces generally do not depend on a polarity of the electric field. Therefore, motion of the particle in response to dielectrophoretic forces does not result from a polarity of the particles but rather from a magnitude of the electric field. Generally, most cells and particles exhibit either pDEP or nDEP at a given frequency.

### SUMMARY

The present invention provides, in some embodiments, a device, kit and method, for shortening the culture-enrichment step that is necessary for detection of rare pathogens in food and any liquid media.

In one aspect of the invention, there is provided an apparatus comprising: a fluid chamber comprising a wall shaped and sized for receiving a fluid sample suspected of containing a microorganism; the fluid chamber is characterized by a minimal width of about 0.6 mm and by a height between about 10 and about 50 μm; comprises a first region and a second region downstream and in fluid communication with the first region, wherein a cross-section of the first region is greater than a cross-section of the second region; a first electrode, electrostatically coupled to the first region, and configured to generate a positive dielectrophoresis (pDEP) force at a first location within the first region; a second electrode, electrostatically coupled to the second region, and configured to generate a pDEP force at a second location within the second region.

In another aspect, there is provided a method for detecting a microorganism within a fluid sample, the method comprising:
i. applying AC to the first electrode and to said second electrode of the apparatus of the invention, to generate pDEP;
ii. introducing the fluid sample into the apparatus while inducing a flow of said fluid sample within the fluid chamber, wherein in response to pDEP the microorganism is retained (i) at the first location, (ii) at the second location, or both (i) and (ii), thereby inducing a flow of the fluid sample within the fluid chamber;
iii. extracting said microorganism from the fluid chamber by introducing a predefined volume of an aqueous solution into the apparatus, thereby obtaining a solution; and
iv. subjecting said solution to an analytical method suitable for detecting the presence of the microorganism within the solution; wherein said fluid sample is suspected of containing the microorganism and is substantially devoid of a particulate matter with a particle size above 15 μm; and wherein a volume of said solution is at least 10 times lower than volume of the fluid sample.

As provided herein, pathogen detection may be significantly improved by incorporating a step of cells concentration based on dielectrophoresis (DEP), so as to reduce the duration of pathogen enrichment step due to the expected detection sensitivity by Real-Time PCR with at least an order of magnitude higher sensitivity than non-concentrated pathogens. In some embodiment, the invention provided methods and devices for increased sensitivity using larger tested sample volumes, due to the ability provided herein to concentrate bacterial cells into a substantially smaller volume.

In some embodiments, the invention is based, in part, on dielecotrophoretic concentration of bacteria directly from a culture-enrichment buffer, using a lab-on-a-chip platform. The device, kit and method of the invention may be used for food safety products and services, in order to obtain testing results in shortened time. The device, kit and method of the invention will significantly contribute to consumer's well-being and minimize food recalls, whilst improving production efficiency by early detection of contaminations, decreasing production loss, and shortening ingredients or finished goods holdups.

In some embodiments, the device, kit and method of the invention comprises microfluidics and dielectrophoresis (DEP) technologies for the pre-concentration of bacteria from a culture-enrichment buffer through a sample volume shrinkage. The concentrated bacterial cells may then be specifically detected by specific means, including but not limited to, PCR.

In some embodiments, the device, kit and method of the invention comprises microfluidics and dielectrophoresis (DEP) technologies for the pre-concentration of bacteria from water and liquid media through a sample volume shrinkage. The concentrated bacterial cells may then be specifically detected by specific means, including but not limited to, PCR.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

Exemplary embodiments are illustrated in referenced figures. Dimensions of components and features shown in the figures are generally chosen for convenience and clarity of presentation and are not necessarily shown to scale. The figures are listed below.

FIG. 1: a scheme representing a non-limiting exemplary configuration of the apparatus of the invention (top view).

FIG. 2: is a flowchart of a method according to some embodiments of the invention.

FIG. 3: Positive DEP based trapping of bacteria via the different electrodes embedded in the microfluidic channel. Electrodes array design and resulting image of positive dielectrophoresis (pDEP).

FIG. 4: Positive DEP based preconcentration of Listeria innocua ATCC 33090 in 10× diluted PBS buffer (1.5 mS/cm). Top images: accumulation of bacterial cells along the electrodes. White scale bar=200 μm. Bottom images: red crosses depicting bacterial cells, demonstrating the outstanding difference between control cells (before concentration) and cells preconcentrated by DEP.

FIG. 5: Quantitative pDEP trapping efficiency of Listeria innocua ATCC 33090 in 10× diluted PBS buffer under a flow rate of 1.5 μL/min and collection time of 5 min. umber of counted bacteria cells in a fixed area (160×160 μm2) before (control) and after preconcentration. Right: the resulting enhanced concentration factor, as normalized by number of counted bacteria cells.

FIG. 6: Real-Time PCR calibration curve targeting detection of E. coli ATCC 8739. Real-Time PCR was conducted prior and following nDEP-based concentration of cells. Top left: genomic DNA calibration curve for E. coli ATCC 8739 at doubling dilutions from 108 ng up to 0.05 ng. Top right: above corresponding DNA amounts to the cells run in DEP experiment. Bottom: zoom-in on merging of relevant DNA amounts drawn from the calibration curve with the cells run in DEP experiment. A) cells after DEP concentration corresponding to 3.4 ng of DNA; vs B) control cells before DEP concentration corresponding to 0.4 ng of DNA.

FIG. 7: Real-Time PCR Quantity Mean graph targeting detection of bacterial 16S rRNA, both Gram-positive and Gram-negative. Real-Time PCR was conducted prior and following nDEP-based concentration of cells. Analysis of the tested triplicate replicas is based on StepOnePlus™ Software v2.3, producing Quantity Mean data based on Ct (cycle threshold) of a DNA calibration curve parameters: the target quantity in each bacterial sample is in comparison to the target quantity in the DNA reference sample. The smaller the Ct, the higher the Quantity Mean value representing an earlier and enhanced detection.

FIG. 8 presents a non-limiting scheme of an exemplary apparatus of the invention in a form of a chip. The chip consists of an array of interdigitated DEP electrodes embedded on both top and bottom glass substrates and a sandwiched thin spacer with channel. This single sandwiched DEP chip can be extended to multi-stacked chips to increase flow rate by channel volume, maintaining the trapping efficiency. The bottom inset shows the fabricated prototype of assembled DEP chip.

FIGS. 9A-B: Detailed components of the single-stacked structure of an exemplary apparatus. FIG. 9A represents a schematic top view of a non-limiting configuration of an exemplary apparatus. FIG. 9B represents a detailed schematic top view and images of: 1: bottom substrate with embedded DEP electrode array, 2: a spacer including microchannel structure, 3: top plate including embedded DEP electrode array and inlet/outlet holes, 4: 1^{st} wide channel zone of the main channel including 1^{st} DEP electrode array, 5: a sandwiched DEP electrode array at 1^{st} channel zone, 6: 2^{nd} channel zone of the main channel including 2^{nd} DEP electrode array, 7: a sandwiched DEP electrode array at 2^{nd} channel zone, 8: waste channel, 9: collecting channel, 10: electric pad for connecting external electric signal, 11: 1^{st} DEP electrode array on the bottom substrate, 12: 2^{nd} DEP electrode array on the bottom substrate 13: 2^{nd} DEP electrode array on the top plate, 14: 2^{nd} DEP electrode array on the top plate, 15: inlet holes, 16: outlet holes. Bottom right image: zoom-in on different critical area of the device, as used in advanced prototypes as well.

FIG. 10: Timelapse images showing positive DEP based preconcentration of Listeria innocua ATCC 33090 using a device with sandwiched embedded electrode arrays. The bacterial cells were spiked in 10× diluted in Brain Heart Infusion Broth (BH enrichment buffer, at 1.0 mS/cm conductivity). The applied flow rate is 8 to 10 μL/min using capillary force by absorbent pad at waste reservoir.

FIG. 11: Real-time PCR amplification plot for Listeria detection in apple juice using DEP. The difference between the enhanced signal resulting from processing the sample on the chip (After DEP) was significantly more sensitive in comparison with the signal without such processing (No DEP) (bottom FIG.). In order to calculate the enhancement factor more precisely and at lower CFU/mL concentrations, samples were diluted 1/10. The resulting enhancement factor, based on Quantity Mean derived from the qPCR software algorithm, was 97 times more sensitive owing to DEP processing. The calibration curves (indicated by arrows pointing down) refer to spiking six different serial dilutions of concentrations of Listeria (at CFU/mL) in LEB solution (no apple juice) as reference.

FIGS. 12A-B: Total count detection in milk using DEP. (12A) Real-time PCR amplification plot of bacterial cells in milk with/without DEP operation and (12B) their normalized PCR signal vs the spiked bacteria concentrations. Two different serial dilutions of concentrations of bacterial cells (10\hat{ }5 and 10\hat{ }6 at CFU/mL) were spiked into the milk solution (1:10 diluted in BH buffer with low conductivity) for DEP. The dot lines with small symbols at (12a) and the dotted line of PCR signals at (12b) are based on spiking of known concentrations of bacteria cells in BH solution (no milk) as reference. At the ‘no DEP’ tests, PCR signals were undetected, due to PCR inhibition by milk. At the ‘DEP’ tests, the obtained PCR signals show a distinct improvement in detection at ˜2 orders of magnitude in comparison to the reference (no milk).

FIGS. 13A-C: Combination of the inertial microfluidic-based pre-filtering and DEP-based bacterial preconcentration/purification in food components. (13a) a schematic presentation showing the sequential steps of the procedure for sample pre-treatment using both inertial microfluidics and DEP-based preconcentration/purification. (13b) representative microscopic image of an inertial microfluidic chip (from Chipshop) showing the sorting of large-sized food components (egg pasta, in this case) into outlet channel 2 to 7, while bacteria cells (E. coli) and small sized (2˜5 μm in diameter) food components move to ends of outlet channels (1 and 8) under the application of a high flow rate flow (1.5 mL/min). (13c) resulting microscopic images of the collected samples solution at each outlet channel and inlet channel.

FIG. 14: Total count detection in of bacteria in egg pasta using DEP. Real-time PCR amplification plot, showing the difference between the enhanced signal resulting from processing the sample on the chip (After DEP, or ‘D’) in comparison with the signal without such processing (No DEP, or ‘C8’), based on the DNA calibration run in parallel. Arrows indicate different tests: ‘D’=after DEP; ‘C8’=sample collected from inertial channel #8; ‘A-sup’=supernatant from stomacher; ‘B’=after 40 μm cell strainer; ‘C5’=sample collected from inertial channel #5; ‘C2’=sample collected from inertial channel #2. The calibration curves (also indicated by arrows) refer to spiking of bacteria in BH solution (no egg pasta).

FIGS. 15A-C: Total count detection in various food types, using a combination of inertial and DEP chips. (15a) Real-time PCR amplification plot, showing the difference between the enhanced signal resulting from processing the sample on the chip (After DEP) in comparison with the signal without such processing (No DEP), based on the DNA calibration run in parallel when testing wheat flour. (15b) Real-time PCR amplification plot of bacterial cells in broiler feed with/without DEP operation and (15c) their normalized PCR signal vs the spiked bacteria concentrations. The dotted line of PCR signals at (15c) is based on spiking of known concentrations of bacteria cells in BH solution (no broiler feed) as reference.

### DETAILED DESCRIPTION

The present invention provides, in some embodiments, a device, kit and method, for shortening the culture-enrichment step that is necessary for detection of rare pathogens in food as well as increasing bacterial detection from water or any fluid sample.

In some embodiments, the invention is based, in part, on a finding that by introducing a sufficient positive dielectrophoresis (pDEP) in a liquid media, it is possible to retain microorganisms (e.g. bacteria) within the region of pDEP. Further, the retained microorganisms are released from the pDEP region and eluted using a small volume of an aqueous solution (usually about 1-10 ul) resulting in in-situ pre-concentration of microorganisms. It is well-known that several food products contain constituents (e.g. enzymes) inhibiting PCR-based RNA/DNA detection. For example, milk constituents significantly interfere with the PCR reaction, resulting in an unacceptably high lowest detection limit of the pathogen in milk. Accordingly, microbial contamination tests of milk or milk products require expensive and time-consuming step of DNA extraction. Whereas the method/apparatus of the invention inducing pre-concentration together with removal of food constituents interfering with PCR-based RNA/DNA detection (also used herein as ‘Inhibitors”) allows the detection of microorganisms (e.g. pathogens) with enhanced sensitivity, compared to conventional methods. It is possible to detect the presence of a pathogen directly from a culture-enrichment buffer, using a lab-on-a-chip platform (i.e. the apparatus of the invention).

The device, kit and method of the invention may be used for food safety products and services, in order to obtain testing results in shortened time. The device, kit and method of the invention will significantly contribute to consumer's well-being and minimize food recalls, whilst improving production efficiency by early detection of contaminations, decreasing production loss, and shortening ingredients or finished goods holdups.

In some embodiments, the device, kit and method of the invention comprises microfluidics and dielectrophoresis (DEP) technologies for the pre-concentration of bacteria from a liquid food sample (or from a culture-enrichment buffer) through a sample volume shrinkage. The concentrated bacterial cells may then be specifically detected by specific means, including but not limited to, PCR, sequencing, etc.

According to some embodiments, there is provided a method for detecting a microorganism within a fluid sample, the method comprising:
i. applying AC to the first electrode and to the second electrode of the apparatus of the invention, thereby generating the pDEP
ii. introducing the fluid sample (e.g., a food product) suspected as having the microorganism into the apparatus, thereby inducing a flow of the fluid sample within the fluid chamber;
iii. introducing a predefined volume of a liquid into the apparatus under conditions suitable for extracting the microorganism from the apparatus, thereby obtaining a solution; and
iv. subjecting the solution to an analytical method suitable for detecting the presence of the microorganism within the solution.

According to some embodiments, there is provided an apparatus comprising a fluid chamber comprising a wall shaped and sized for receiving a fluid sample suspected of containing a microorganism; the fluid chamber comprises a first region and a second region downstream and in fluid communication with the first region, wherein a cross-section of the first region is greater than a cross-section of the second region; a first electrode, electrostatically coupled to the first region, and configured to generate a positive dielectrophoresis (pDEP) force at a first location within the first region; and optionally a second electrode, electrostatically coupled to the second region, and configured to generate a pDEP force at a second location within the second region.

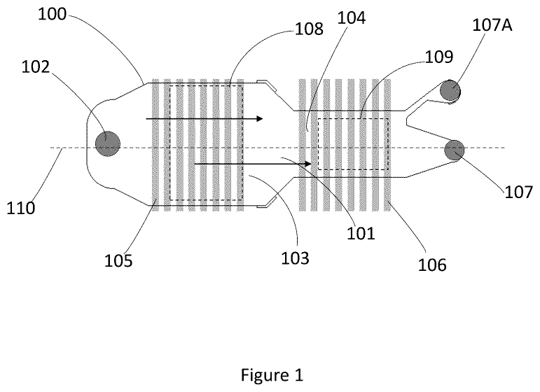

Reference is now made to FIG. 1 representing a top-view of a non-limiting exemplary apparatus according to the present invention. The apparatus 100 may comprise a fluid chamber 101 comprising at least one wall and configured for containing a fluid volume (i.e. for receiving a fluid sample). Optionally the fluid chamber 101 has a longitudinal axis 110. The apparatus 100 may comprise in inlet 102 configured for receiving a fluid sample. The inlet 102 is in fluid communication with the fluid chamber 101. Optionally, the inlet 102 is in operable communication with a pump. Optionally, the inlet 102 is configured for injection of the fluid sample into the apparatus 100. Optionally, the inlet 102 is configured for transferring the fluid sample into the fluid chamber 101.

In some embodiments, the fluid chamber 101 is in a form of a channel characterized by any one of a length dimension between about 10 mm and 10 cm; and by a height dimension between about 10 and 50 μm. In some embodiments, the fluid chamber 101 is characterized by a width of at least about 0.6 mm, or between 0.5 and about 2 mm. In some embodiments, the apparatus 100 is configured to support a flow of the fluid sample within the fluid chamber 101. In some embodiments, the flow is directed along and parallel to the longitudinal axis 110. In some embodiments, the flow is along the longitudinal axis 110 and directed from the inlet 102 towards the outlet 107. The flow direction is assigned by black arrows.

Optionally, the fluid chamber 101 may comprise a first region 103 in fluid communication with a second region 104. Optionally, the second region 104 is downstream to the first region 103, wherein the second region 104 is characterized by a width lower than the width of the first region 103.

The apparatus 100 may comprise a first electrode 105 (optionally in a form of an electrode array). The first electrode 105 is positioned on or within at least one wall and is located in the first region 103. Upon applying AC, the first electrode 105 is configured to generate a DEP (pDEP) in the fluid volume in close proximity to the first electrode 105. Upon applying AC, the first electrode 105 is configured to generate a DEP (pDEP) in the first location 108.

The apparatus 100 may comprise a second electrode 106 (optionally in a form of an electrode array). The second electrode 106 is positioned on or within at least one wall and is located in the second region 104. Upon applying AC, the first electrode 106 is configured to generate a DEP (pDEP) in the fluid volume in close proximity to the second electrode 106. Upon applying AC, the second electrode 106 is configured to generate a DEP (pDEP) in the second location 109. The first electrode 105 and the second electrode 106 may be positioned (i) at a bottom wall of the fluid chamber 101, (ii) at a top wall of the fluid chamber 101, or both (i) and (ii).

The apparatus 100 may comprise a plurality of fluid chambers and a plurality of the first and second electrodes, wherein each of the plurality of fluid chambers is in fluid communication with each other. Such configuration refers herein as a multi-stack configuration and has been successfully implemented by the inventors.

The apparatus 100 may comprise an outlet, such as a first outlet 107 (e.g. for the collection of the sample), and further optionally a second outlet 107A (e.g. for the collection of the waste fluid).

According to some embodiments, the apparatus of the invention comprises a microchannel optionally comprising an inlet reservoir (configured for receiving a fluid sample), and further comprises means for applying dielectrophoretic (pDEP) (e.g. an electrode array), and optionally a collecting chamber and optionally a waste chamber.

The terms “channel” and “microchannel” are used herein throughout and may comprise or be adjacent to microelectrodes, and/or related control systems.

The term “microchannel” as used herein refers to a groove or plurality of grooves created on a suitable substrate with at least one of, and optionally, all of the dimensions of the groove being, without limitation, in the micrometer range, e.g., 1 μm to 1000 μm. In some embodiments, the height is micro sized.

In some embodiments, the fluid chamber is defined by at least one wall. In some embodiments, the fluid chamber comprises a plurality of walls. In some embodiments, the fluid chamber is a microfluidic chamber, characterized by a height dimension between 10 and 100 μm, between 10 and 50 μm, between 1 and 50 μm, between 5 and 50 μm, between 10 and 30 μm, between 1 and 30 μm, between 1 and 40 μm, including any range between.

In some embodiments, the fluid chamber is characterized by a height dimension as disclosed herein above and further characterized by at least one of: a width dimension between about 0.6 and about 2 mm, about 0.6 and about 1.8 mm, between about 0.4 and about 2 mm, between about 0.5 and about 2 mm, between about 0.5 and about 5 mm, between about 0.5 and about 10 mm, between about 0.5 and about 1 mm, between about 0.5 and about 3 mm, including any range between; and a length dimension between about 5 mm and 10 cm, between 10 mm and 10 cm, between about 10 mm and 50 cm, between about 10 mm and 3 cm, between about 10 mm and 5 cm, between about 5 mm and 10 cm.

In some embodiments, the fluid chamber is characterized by ratio of the width dimension to the length dimension between 1:2 and 1:10000, between 1:2 and 1:5, between 1:2 and 1:10, between 1:10 and 1:20, between 1:20 and 1:50, between 1:50 and 1:100, between 1:100 and 1:1000, between 1:100 and 1:10000, between 1:100 and 1:500, between 1:500 and 1:1000, between 1:1000 and 1:2000, between 1:2000 and 1:5000, between 1:5000 and 1:10000, including any range or value therebetween.

Optionally, the fluid chamber is configured to hold a fluid volume, wherein the fluid volume refers to the volume of the fluid sample within the fluid chamber. Optionally, the fluid chamber is configured for handling a fluid volume (the fluid sample) having a volume within a microliter and/or a milliliter range. Optionally, the fluid chamber has a volume between 100 ul and 100 ml, between 100 ul and 10 ml, between 100 ul and 1 ml, between including any range between.

The term “chamber”, as used herein, means a natural or artificial enclosed space or cavity known to those of skill in the art. By “enclosed”, it is further meant to refer to at least partially enclosed.

In some embodiments, the fluid chamber has a rectangular shape. In some embodiments, the microfluidic chamber has a circular shape. In some embodiments, the fluid chamber has a semicircular shape. In some embodiments, the fluid chamber has an ellipsoid shape.

In some embodiments, the length dimension of the fluid chamber is greater than the width dimension by about 2, 5, 10, 20, 30, 50, 70, 80, 100, 200, 300, 500 times including any range between. In some embodiments, the fluid chamber is in a form of a plurality of channels. In some embodiments, the fluid chamber comprises a plurality of chambers interconnected with each other (e.g. in fluid communication, so as to allow flow of the fluid from one chamber to a subsequent chamber).

In some embodiments, the fluid chamber comprises a first region and a second region downstream and in fluid communication with the first region. In some embodiments, a cross-section of the first region is greater than a cross-section of the second region. In some embodiments, the second region is downstream of the first region, and the second region and first region are located along a longitudinal axis of the fluid chamber. In some embodiments, a ratio between the cross-section of the first region to the cross-section of the second region within the fluid chamber is between 1:1.5 and 1:10, between 1:2 and 1:10, between 1:2 and 1:5, between 1:2 and 1:3, between 1:1.5 and 1:3, including any range between.

In some embodiments, the fluid chamber is configured to support a flow of the fluid sample (or of the fluid volume). In some embodiments, the flow is along the longitudinal axis of the fluid chamber. In some embodiments, the flow refers to the flow rate in the first region and/or within the second region. In some embodiments, the flow direction is from the inlet towards the outlet along the longitudinal axis of the fluid chamber. It is postulated that the maximum flow rate is selected so as to prevent damage to the microorganism. In some embodiments, the maximum flow rate is so as to retain at least 90%, at least 95%, at least 97%, or between 90 and 100%, between 90 and 95% of the initial count (e.g. CFU) of the microorganism. The term “initial count” or “initial loading” each independently refer to the total count/loading in the fluid sample before introducing thereof into the apparatus of the invention. In some embodiments, the flow is at a rate between 10 and 50 ul/min, between 5 and 50 ul/min, between 20 and 50 ul/min, between 30 and 50 ul/min, including any range between.

In some embodiments, the wall consists essentially of a dielectric material. In some embodiments, the dielectric material is characterized by an electrical resistivity of at least 1 MΩ, or at least 10 MΩ. The term “dielectric material” encompasses the broad expanse of nonmetals considered from the standpoint of their interaction with electric, magnetic, or electromagnetic fields such that the materials are capable of storing electric energy. A dielectric material is a substance that is a poor conductor of electricity, but an efficient supporter of electrostatic fields.

In some embodiments, the dielectric material comprises any one of: glass, ceramics (e.g. titania, Ta₂O₅, silicon oxonitride (SiON), silicon oxide, alumina, silicon nitride), a polymeric material (e.g. comprising a synthetic organic polymer, such as polyacrylate, polycarbonate, PVC, polyolefin, poly-dimethylsiloxane (PDMS), or any combination and/or copolymer thereof. In some embodiments, the wall consists essentially of PDMS or a glass substrate.

In some embodiments, the wall has a sufficient mechanical strength and/or thickness to support the flow. In some embodiments, the wall is characterized by a thickness of at least 5 μm, at least 10 μm, at least 15 μm, at least 20 μm, at least 30 μm, at least 50 μm, at least 100 μm, between 10 and 500 μm, between 10 and 100 μm, between 50 and 500 μm, between 50 and 200 μm, between 30 and 500 μm, including any range between.

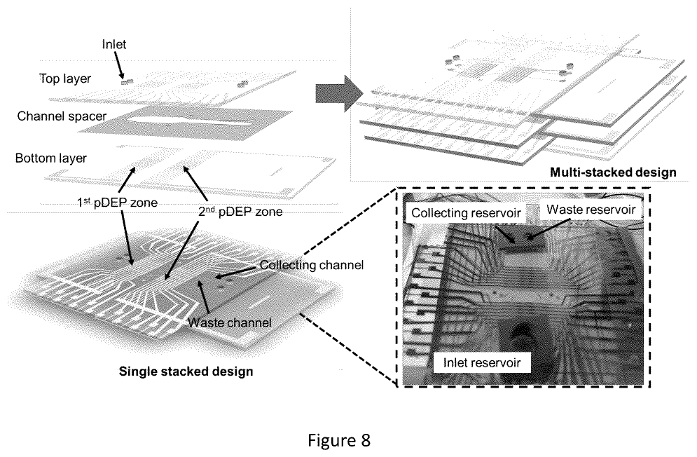

In some embodiments, the fluid chamber is defined by a single wall, or comprises a plurality of walls (e.g., 2, 3, 4 walls). In some embodiments, the first electrode and the second electrode are located on top or within (e.g. embedded within the wall) at least one wall of the microfluidic chamber. In some embodiments, the first electrode and the second electrode are located on or within the top wall and the bottom wall of the fluid chamber. In some embodiments, the apparatus comprises a plurality of walls positioned parallel to each other and coupled to the fluid volume. In some embodiments, the plurality of walls are positioned along and perpendicular to the vertical axis of the fluid chamber. Such configuration may be preferential for fluid chambers with a height above 10 um, such as between 150 um and 10 mm, between 100 um and 10 mm, or between 150 um and 5 mm, including any range between, resulting in an increased volume of the fluid chamber, which might be suitable for increased through-put. In some embodiments, the plurality of walls form multi-stack fluid chambers, wherein fluid chambers are in fluid communication to each other, operate in parallel and have common inlets (such as schematically presented in FIG. 8).

In some embodiments, the first electrode and the second electrode face the liquid volume. In some embodiments, the first electrode and the second electrode are each independently configured to receive an alternating current (AC). In some embodiments, the first driving electrode are in operable communication with an AC source. In some embodiments, the first electrode and the second electrode affixed to at least one wall.

In some embodiments, the first electrode and the second electrode are configured to generate a pDEP across the fluid volume. In some embodiments, the first electrode and the second electrode are configured to generate a pDEP at a predetermined location within the fluid volume. In some embodiments, the predetermined location is at close proximity to the first electrode and/or to the second electrode. In some embodiments, the first electrode and the second electrode are electrically coupled to the fluid volume. In some embodiments, the first electrode and the second electrode are electrically coupled to the fluid volume, so as to generate a pDEP at a predetermined location within the fluid volume. In some embodiments, the first electrode and the second electrode are configured to generate pDEP in the entire depth of the fluid volume at the predetermined location.

In some embodiments, the term “entire depth” as used herein, refers to at least 60%, at least 70, at least 80%, at least 90%, between 60 and 99%, between 60 and 100%, between 70 and 99%, between 80 and 100%, between 90 and 100%, between 90 and 99% of the fluid volume depth at the predetermined location, including any range between.

In some embodiments, the first electrode and the second electrode are configured to generate a sufficient pDEP force to substantially retain the microorganism in the predetermined location. In some embodiments, the term “substantially retain” encompasses retention of at least 90%, at least 95%, at least 97%, at least 99%, or 100%, between 90 and 100%, between 90 and 95%, between 90 and 99% of the initial count of the microorganism within the fluid sample. In some embodiments, the height dimension of the fluid chamber is predestined by the effective pDEP force, wherein the effective pDEP force is so as to attract the microorganisms to the electrode. In some embodiments, the effective pDEP force is so as to substantially retain the microorganism in the predetermined location.

In some embodiments, the pDEP force induced by the first electrode and/or by the second electrode is sufficient for attracting the microorganism (e.g. one or more bacteria, such as a gram-positive and/or gram-negative bacteria) to the first electrode, and/or to the second electrode.

In some embodiments, the first electrode is located within the first region of the fluid chamber, and is configured to generate a pDEP at a predetermined location within the first region (also referred to herein as “the first location”). In some embodiments, the first electrode is configured to generate a pDEP within the entire depth of the fluid volume at the first location.

In some embodiments, the second electrode is located within the second region of the fluid chamber and is configured to generate a pDEP at a predetermined location within the second region (also referred to herein as “the second location”). In some embodiments, the second electrode is configured to generate a pDEP within the entire depth of the fluid volume at the second location. In some embodiments, the second electrode is located downstream to the first electrode. In some embodiments, the second electrode is located distal to the inlet, and proximal to the outlet. In some embodiments, the second electrode is located upstream to the outlet. Within being limited to any particular theory or mechanism, second electrode is located close to the outlet, so as to minimize the pathway of a microorganism from the second electrode until it reaches the outlet during the extraction step. In order to minimize the volume of the solution (see Method section), it is preferable to locate the second electrode proximal to the outlet, thereby minimizing the volume of the aqueous solution required for the extraction of the microorganism.

The apparatus typically includes at least one electrode (e.g., a pair of sorting electrodes, or an electrode array configured to induce pDEP) in in operable communication with an electric source configured to apply alternating current (AC) field frequencies to drive the at least one electrode to generate pDEP force in the chamber. The apparatus may be configured and shaped to define, in response to the pDEP, force to manipulate the bacterial cells such that they are attracted to the electrode and brought into proximity to the electrode exhibiting a pDEP response.

In some embodiments, each of the first electrode and the second electrode are in a form of an array of electrodes. In some embodiments, the first electrode and the second electrode are positioned within the same plane. In some embodiments, the first electrode and the second electrode are in a form of substantially parallel arrays of electrodes, positioned perpendicular to the longitudinal axis of the fluid chamber. In some embodiments, the array of electrodes is characterized by a length along the longitudinal axis of the fluid chamber ranging between 10 um and 10 mm, between 100 um and 10 mm, between 100 um and 1 mm, between 100 um and 2 mm, between 100 um and 5 mm, including any range between.

In some embodiments, the array of electrodes is characterized by at least one of (i) a width of each of the parallel arrays is independently between 1 and 50 um, between 1 and 30 um, between 1 and 20 um, between 5 and 50 um, between 5 and 20 um, between 10 and 50 um, between 10 and 20 um, including any range between; and (ii) a distance between the adjacent parallel arrays is independently between 1 and 50 um, between 1 and 30 um, between 1 and 20 um, between 5 and 50 um, between 5 and 20 um, between 10 and 50 um, between 10 and 20 um, including any range between.

In some embodiments, the array of electrodes comprises between 10 and 500, between 5 and 20, between 10 and 20, between 50 and 200, between 100 and 500, between 100 and 200 pairs of electrodes, including any range between.

In some embodiments, each array is independently controlled. In some embodiments, each electrode within the electrode array is independently controlled. In some embodiments, the one or more electrodes/array is controlled via a control unit (CU).

Herein, the term “array of electrodes” may refer to a single electrode or a plurality of electrodes. The terms “electrodes”, “array of electrodes” or “arrangement of electrodes” do not necessarily refer to any specific geometric arrangement of electrodes.

Non-limiting exemplary electrodes are selected from a carbon electrode, or a metal electrode, wherein the metal is selected from copper, gold, silver, nickel, zinc, antimony, bismuth, iridium, and platinum, including any alloy or mixture thereof.

According to some embodiments, the embedded electrode arrays for applying pDEP are located either on the bottom substrate or on both top and bottom substrates to enhance DEP force, allowing higher flow rate (8˜10 μL/min).

According to some embodiments, the embedded electrode arrays for applying pDEP are multi-stacked with common inlets for high-throughput preconcentration of bacterial cells.

According to some embodiments, there is provided a device comprising of serial microchannels of different geometries as a means for efficient collection of the preconcentrated bacterial cells within a small volume so as to increase volume reduction.

According to some embodiments, there the microchannel comprises a microfluidic layer with electrodes embedded either on the bottom or on both top and bottom walls of the channel. According to some embodiments, the microchannel is a single stack microchannel.

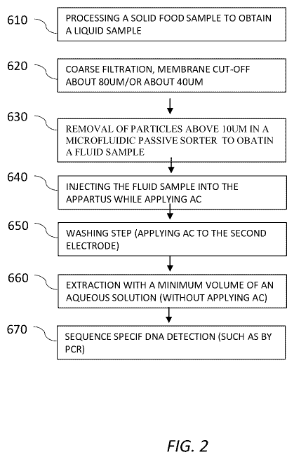

An exemplary configuration of the electrode arrays is illustrated by FIGS. 2, and/or 9.

In some embodiments, the apparatus comprises an inlet suitable to receive the fluid sample and in fluid communication with the fluid chamber. In some aspects, the fluid chamber is shaped to define a main flow channel shaped to define an inlet for introducing the food sample into the main flow channel and shaped and sized for flow of the sample therethrough. The electrode array is disposed in the main flow channel downstream from the inlet, and comprises electrodes each independently controllable by a control unit, so as to generate pDEP sufficient for attraction and trapping of the microorganism at the predefined location (e.g. the first or the second location, as disclosed herein). The fluid chamber is further shaped to define a first outlet (for sample collection) in fluid communication with the second region, and downstream to the second electrode. The fluid chamber is further shaped to define a second outlet (for waste collection) in fluid communication with the second region, and downstream to the second electrode.

In some embodiments, the first outlet and the second outlet are in fluid communication with the fluid chamber (e.g. the second region) via separate channels. In some embodiments, the first outlet is in fluid communication with the fluid chamber (e.g. the second region) via a first channel. In some embodiments, the second outlet is in fluid communication with the fluid chamber (e.g. the second region) via a second channel. In some embodiments, the liquid flow from the second region is controllable to reach the first outlet or the second outlet. In some embodiments, the second region comprises a valve configured to direct the flow from the second region into the second channel, or into the first channel.

In some embodiments, the inlet is adopted for inserting a syringe (e.g. via connection having a shape and dimension compatible with the syringe). In some embodiments, the inlet is characterized by a volume of at least 50 μL, at least 100 μL, at least 500 μL, at least 1000 μL, at least 2 ml, at least 10 ml, between 50 μL and 10 ml, between 50 μL and 2 ml, between 50 μL and 1 ml, including any range between.

In some embodiments, the apparatus comprises an outlet in fluid communication with the fluid chamber. In some embodiments, the outlet is configured to receive a liquid form the fluid chamber. In some embodiments, the outlet is configured to receive a solution, wherein the solution is as described hereinbelow. In some embodiments, the outlet comprises a first outlet configured for collecting the sample, and a second outlet configured for collecting a waste liquid. In some embodiments, the outlet is characterized by a volume of between 1 μL and 1 ml, between 1 μL and 100 μL, between 1 μL and 50 μL, between 1 μL and 500 μL, between 1 μL and 10 μL, including any range between.

In some embodiments, the apparatus of the invention is configured for concentrating (e.g. in-situ concentrating) the fluid sample. In some embodiments, upon inducing pDEP by the first electrode and by the second electrode, the apparatus is configured for entrapment of at least one microorganism cell within the first location, or within the second location. In some embodiments, upon introducing a predefined volume of a liquid the apparatus of the invention is configured for subsequent extraction of the microorganism from the fluid chamber, to obtain a solution comprising the microorganism. In some embodiments, the concentration of the microorganism within the solution is at least 10 times, at least 50 times, at least 100 times, at least 500 times, at least 1000 times, at least 10000 times greater than the initial concentration in the fluid sample. In some embodiments, a volume of the solution is between 1 μL and 500 μL, between 1 μL and 100 μL, between 1 μL and 50 μL, between 1 μL and 10 μL, including any range between.

In some embodiments, the volume of the solution is lower than the volume of the fluid sample by at least 10 times, at least 50 times, at least 100 times, at least 500 times, at least 1000 times, at least 10000 times, between 10 and 1000 times, including any range between.

In some embodiments, the apparatus of the invention is configured for preconcentrating the fluid sample, prior to subjecting the fluid sample to an analytical method suitable for detecting the presence of the microorganism within the sample. In some embodiments, the apparatus of the invention is configured for preconcentrating the fluid sample, prior to subjecting the fluid sample to PCR. In some embodiments, the apparatus of the invention is configured for preconcentrating the fluid sample, and removing fodd constituents which may potentially interfere with PCR (used herein as “inhibitors”), thereby enhancing the sensitivity (i.e. reducing the lowest detection limit, LOD) of the PCR by about 1, 2 or 3 orders of magnitude, compared to a similar sample which has not been treated by the apparatus of the invention. Moreover, a sample treated by the apparatus of the invention substantially retains the initial total count of the microorganisms, as described herein. In some embodiments, the apparatus of the invention is configured for processing a liquid food sample, to obtain a solution (i.e. an aqueous sample) substantially devoid of inhibitors, and characterized by an enhanced microorganism concertation, wherein enhanced is by at least about 10 times, at least about 20 times, at least about 50 times, at least about 100 times, at least about 500 times, at least about 1000 times, at least about 10.000 times, between 10 and 1000 time, between 10 and 10.000 times greater concertation, compared to the initial microorganism concertation within the non-processed liquid food sample.

Accordingly, using the apparatus of the invention is a prerequisite for an efficient detection for the presence of a microorganism in a food sample, with a LOD of at least 1 CFU/ml, or at least 10 CFU/ml.

According to some embodiments, the microorganisms are concentrated (by introducing the fluid sample comprising thereof into the apparatus of the invention) from an enrichment buffer, followed by a PCR detection using a specific PCR mix for amplification of a specific target microorganism (e.g. pathogenic bacteria).

According to some embodiments, the microorganisms are preconcentrated from a liquid food and/or water sample followed by a PCR detection According to some embodiments, the PCR detection is performed using either a specific PCR mix for amplification of a specific target microorganism or a PCR mix intended for amplification of several bacteria types (both Gram-positive and Gram-negative) for bacterial total count purposes.

In some embodiments, the apparatus of the invention is configured to process a fluid sample characterized by a sufficient electrical conductivity. In some embodiments, the fluid sample is a liquid sample. In some embodiments, the fluid sample is an aqueous sample. In some embodiments, the fluid sample is an aqueous dispersion, an aqueous solution, or aqueous suspension. Optionally, the fluid sample is a Newtonian liquid (fluid). As used herein and in the art, Newtonian liquid is a fluid in which the viscous stresses arising from its flow, at every point, are linearly proportional to the local strain rate—the rate of change of its deformation over time. In some embodiments, the fluid sample is a non-Newtonian liquid (fluid).

In some embodiments, the fluid sample is suspected of containing a microorganism. In some embodiments, the microorganism is a pathogenic microorganism. In some embodiments, the total count of the microorganism within the fluid sample is at least 1 CFU, at least 10 CFU, at least 5 CFU, at least 30 CFU, at least 50 CFU, at least 100 CFU, including any range between.

In some embodiments, a microorganism loading within the fluid sample is at least 1 CFU/ml, at least 10 CFU/ml, at least 50 CFU/ml, at least 100 CFU/ml, at least 1000 CFU/ml, at least 10000 CFU/ml, including any range between. It should be appreciated that the term “microorganism loading” refers to the minimum concentration of the microorganism(s) detectable by the method of the invention. While it is challenging to detect very low concentrations of microorganisms within a food sample using current microbiological methods, there is no limit with respect to the maximum detectable concentration. Thus, it should be apparent that the maximum microorganism concentration in the fluid sample detectable by the method of the invention is unlimited.

In some embodiments, the microorganism comprises any one of: a bacterial cell, at a yeast cell, and a spore, or any combination thereof. In some embodiments, the microorganism is a pathogenic microorganism. In some embodiments, the microorganism is a pathogenic bacterium, or a spore thereof. In some embodiments, the microorganism is a viable cell, or a dead cell. In some embodiments, the microorganism comprises a single specie or a plurality of distinct species (e.g., 2, 4, 5, 10, 20, or more).

In some embodiments, the fluid sample is characterized by a pH value from 1 to 9, from 2 to 8, from 3 to 8, from 3 to 7, from 3 to 4, from 5 to 7, from 5 to 8, from 3 to 4.5, from 4.5 to 7.5, from 4 to 7, from 5 to 7.5, from 6 to 7.5, from 6 to 8 including any range or value therebetween.

In some embodiments, the fluid sample is characterized by (i) electrical conductivity of at least 0.03 mS/cm, at least 0.05 mS/cm, at least 0.1 mS/cm, or between 5 and 1000 mS/m, between 5 and 500 mS/m, between 5 and 100 mS/m, between 5 and 200 mS/m, between 1 and 1000 mS/m, between 10 and 1000 mS/m, between 50 and 1000 mS/m, between 100 and 1000 mS/m, between 100 and 1000 mS/m, between 200 and 1000 mS/m, including any range between; by (ii) viscosity at 25° C. between 1 and 1000 cP, between 1 and 10 cP, between 1 and 100 cP, between 1 and 500 cP, between 1 and 200 cP, including any range between, or both (i) and (ii). In some embodiments, the fluid sample is an aqueous sample having a viscosity between 1 and 10, or less, and characterized by an electrical conductivity between 0.05 and 2 mS/cm.

In some embodiments, the fluid sample is a liquid food sample (e.g., a beverage). In some embodiments, the fluid sample is derived from a food sample (e.g., any food product including a beverage, or any edible matter). In some embodiments, the fluid sample is derived from a water sample. In some embodiments, the fluid sample is derived from a biological sample (e.g. a microbiological sample), wherein the biological sample comprises a bacteria growth medium or an enrichment buffer.

In some embodiments, the fluid sample is homogenous (e.g. devoid of phase separation). In some embodiments, the term “derived” encompasses any processing of the food sample including, dilution (e.g. dilution of a beverage with an aqueous buffer), cutting, grinding, crushing, blending, enzymatic pre-treatment, including any combination thereof. Various methods for the processing of a food sample are well-known in the art. In some embodiments, the food sample is processed by introducing thereof into a blender (e.g. Stomacher) to obtain the fluid sample.

In some embodiments, a food sample is diluted with a buffer to obtain the fluid sample characterized by a conductivity as disclosed hereinabove.

Some aspects of the present invention include adjusting the sample (e.g., the food sample) and providing a medium having a conductivity suitable for inducing pDEP response at a given frequency. However, if such an adjustment will be done, it will involve a minimal dilution (resulting in reduction of the bacteria concentration) of the sample so as to obtain pDEP response but still get an overall concentration effect relative to the initial sample (taken directly from the culture-enrichment buffer) concentration. For some aspects, the conductivity of the medium in which the bacterial cells are disposed and in which the DEP is generated is 5-1000 mS/m.

Due to the high conductivity of the culture-enrichment buffer all samples following the enrichment step, regardless of their food source, will have approximately the same solution conductivity and hence will have a similar DEP response, thus, saving the need to perform pre-calibration of the operating parameter. In some embodiments, the medium is an aqueous buffer.

In some embodiments, the aqueous buffer has an ionic strength in a range from 100 nM to 500 mM, from 100 nM to 500 nM, from 500 nM to 1 uM, from 1 uM to 100 uM, from 100 uM to 500 uM, from 500 uM to 700 uM, from 700 uM to 1 mM, including any range or value therebetween.

In some embodiments, the aqueous buffer comprises an acid and a conjugate base thereof. In some embodiments, the aqueous buffer comprises a base and a conjugate acid thereof. Non-limiting examples of acids comprise but are not limited to acetic acid, lactic acid, 2-(N-morpholino) ethanesulfonic acid (MES), citric acid, 4-(2-hydroxyethyl)-1-piperazineethanesulfonic acid (HEPES) or a combination thereof.

Non-limiting examples of buffers comprise but are not limited to: acetic acid/NaOH, lactic acid/NaOH, bistris/HCl, tris/HCl, MES/HCl, phosphate buffered solution (PBS), citric acid/NaOH, MES/NaOH, HEPES/NaOH or any combination thereof.

In some embodiments, the fluid sample is substantially devoid of a particulate matter with a particle size above 10 um, above 15 um, above 20 um, above 30 um, or above 40 um. In some embodiments, the fluid sample comprises not more than 1% w/w, not more than 0.1% w/w, not more than 0.01% w/w, not more than 0.001% w/w of particles with a particle size above 10 um, above 15 um, above 20 um, above 30 um, or above 40 um.

Optionally, the disclosed apparatus or system further comprises a computer program product.

### Method

In another aspect, there is provided a method for detecting a microorganism within a fluid sample, the method comprising applying AC to the first electrode and optionally to the second electrode of the apparatus of the invention, to generate pDEP; performing a sample injection by introducing the fluid sample into the apparatus while inducing a flow of the fluid sample within the fluid chamber, wherein in response to the pDEP the microorganism is retained (i) at the first location, (ii) at the second location, or both (i) and (ii); performing extraction by introducing a predefined volume of an aqueous solution into the apparatus under conditions suitable for extracting the microorganism from the fluid chamber, thereby obtaining a solution (e.g. in the outlet); and subjecting the solution to an analytical method suitable for detecting the presence of the microorganism within the solution; wherein the fluid sample is suspected of containing the microorganism. In some embodiments, the fluid sample is as described hereinabove (e.g. is derived from a food sample/biological sample, and is characterized by electrical conductivity of at least 0.05 mS/cm, or between 0.05 and 2 mS/cm). In some embodiments, the predefined volume of the aqueous solution is between 1 and 10 ul, optionally wherein the aqueous solution is a buffer (e.g. a PCR buffer). In some embodiments, the extraction is performed without applying AC to the first electrode and to the second electrode.

In another aspect, there is provided a method for detecting a microorganism within a fluid sample, the method comprises step i. of applying AC to the first electrode and to the second electrode of the apparatus of the invention, to generate pDEP at the first location and at the second location; step ii. (sample injection) of introducing the fluid sample into the apparatus while inducing a flow of the fluid sample within the fluid chamber, wherein in response to the pDEP the microorganism is retained (i) at the first location, (ii) at the second location, or both (i) and (ii); optionally performing a washing step comprising introducing a first aqueous solution into the apparatus while applying AC to the second electrode, thereby inducing a translocation of the microorganism to the second location; performing step iii. (extraction) comprising introducing a predefined volume of a second aqueous solution, thereby extracting the microorganism from the fluid chamber, thereby obtaining a solution (e.g. in the outlet); and step iv. (detection) subjecting the solution to an analytical method suitable for detecting the presence of the microorganism within the solution; wherein the fluid sample is substantially devoid of particles with a particle size above 10 um, or above 15 um; wherein a volume of the solution is at least 10 times lower than volume of the fluid sample; and wherein the fluid sample is suspected of containing the microorganism. In some embodiments, introducing is via the inlet. In some embodiments, the washing step is performed upon completion of the introduction of the fluid sample into the apparatus. In some embodiments, the washing step is performed by applying AC to the second electrode so as to generate pDEP at the second location, without applying AC to the first electrode; thereby translocating the microorganism from the first location to the second location and retaining the microorganism at the second location. In some embodiments, the washing step is for (i) translocating the microorganism, and/or for (ii) substantially removing the constituents of the fluid sample which are not the microorganism.

In some embodiments, the first and/or the second aqueous solution is an aqueous buffer solution. In some embodiments, the second aqueous solution is a PCR buffer. In some embodiments, the volume of the second aqueous solution and/or of the solution (from the extraction step) is between 1 and 10 ul, 1 and 5 ul, 1 and 20 ul, 1 and 50 ul, including any range between. In some embodiments, the first aqueous solution characterized by electrical conductivity of at least 0.05 mS/cm, or between 0.05 and 2 mS/cm.

In another aspect, there is provided a method for detecting a microorganism within a fluid sample suspected of containing the microorganism, the method comprises:
step i. comprising applying AC to the first electrode and to the second electrode of the apparatus of the invention, to generate the pDEP at the first location and at the second location;
step ii. (sample injection) comprising introducing the fluid sample into the apparatus while inducing (e.g. via a pump or by capillary force) a flow of the fluid sample within the fluid chamber at a flow rate between about 10 and about 50 μl/min, wherein in response to the pDEP the microorganism is retained (i) at the first location, (ii) at the second location, or both (i) and (ii);
washing step comprising introducing a first aqueous solution into the apparatus, and generating a flow the first aqueous solution within the fluid chamber at a flow rate between about 5 and about 30 μl/min, while applying AC to the second electrode without applying AC to the first electrode, thereby inducing a translocation of the microorganism to the second location; wherein the first aqueous solution is characterized by electrical conductivity of at least 0.05 mS/cm, or between 0.05 and 2 mS/cm, and wherein a volume of the first aqueous solution is at least one fluid volume (corresponding to the volume of the fluid chamber);
step iii. (extraction) comprising introducing a predefined volume of the second aqueous solution into the apparatus, and generating a flow the second aqueous solution within the fluid chamber to extract the microorganism from the fluid chamber into the outlet, thereby obtaining the solution (e.g. in the first outlet), wherein a volume of the second aqueous solution is between 1 and 10 ul; and
step iv. (detection) of and subjecting the solution to an analytical method suitable for detecting the presence of the microorganism within the solution, wherein a volume of the solution is at least 10 times lower than volume of the fluid sample. In some embodiments, the steps i.-iv. are executed in a subsequent order. In some embodiments, the solution consist essentially of the microorganism dispersed within the aqueous solution. In some embodiments, the solution is substantially devoid of food constituents (e.g. inhibitors).

In some embodiments, the fluid sample is as described hereinabove. In some embodiments, the method of the invention comprises step i. of generating pDEP within at least one predetermined location in the fluid chamber. In some embodiments, the step i. comprises generating pDEP within the first location. In some embodiments, the step i. comprises generating pDEP in the entire depth of the fluid volume within the first location and/or within the second location, as described herein. In some embodiments, the pDEP generated in step i. is sufficient for retaining the microorganism. In some embodiments, the pDEP generated in step i. is sufficient for retaining the microorganism at the first location (i.e. within the fluid volume in above or in close proximity to the first electrode). In some embodiments, the pDEP generated in step i. is sufficient for retaining the microorganism at the second location (i.e. within the fluid volume in above or in close proximity to the second electrode).

In some embodiments, the step i. comprises providing the apparatus of the invention and applying AC to the first electrode and optionally to the second electrode, wherein the AC is sufficient for inducing pDEP in the predetermined location.

In some embodiments, the step ii. (sample injection step) comprises introducing the fluid sample into the apparatus so as to retain the microorganism (i) at the first location, (ii) at the second location, or both (i) and (ii). In some embodiments, the step ii. comprises introducing the fluid sample into the apparatus so as to retain the microorganism (i) at the first location. In some embodiments, the method comprises step ii. comprising introducing the fluid sample into (or contacting the fluid sample with) the apparatus, thereby resulting in retention of the microorganism (i) at the first location, (ii) at the second location, or both (i) and (ii).

In some embodiments, the volume of the fluid sample is up to 5 ml, up to 2 ml, up to 1 ml, between 0.01 and 5 ml, between 0.1 and 2 ml, between 0.01 and 2 ml, between 0.01 and 1 ml, including any range between.

In some embodiments, the fluid sample is introduced into the apparatus with the inlet. In some embodiments, introducing or contacting of the fluid sample is by injecting the fluid sample into the apparatus via the inlet. In some embodiments, step ii. comprising introducing the fluid sample into the apparatus while inducing (e.g. via a pump or by capillary force) a flow of the fluid sample within the fluid chamber at a flow rate between about 10 and about 50 μl/min, between about 2 and about 50 μl/min, between about 2 and about 10 μl/min, between about 10 and about 30 μl/min, between about 30 and about 50 μl/min, including any range between. In some embodiments, the flow is in the first region and in the second region. In some embodiments, step ii. comprises applying a pump (e.g. a syringe pump) to transfer the fluid sample from the inlet into the fluid chamber, and for inducing flow in the liquid chamber as described herein. In some embodiments, the inlet of the apparatus is in operable communication with a pump. In some embodiments, the sample is transferred via a capillary force.

In some embodiments, step ii. comprising inducing a flow of the fluid sample within the fluid chamber, while retaining the microorganism at the first location. In some embodiments, step ii. comprising inducing a flow of the fluid sample within the fluid chamber, while retaining the microorganism at the first location and optionally at the second location. In some embodiments, step ii. comprising inducing a flow of the fluid sample within the first region of the fluid chamber towards the second outlet (while directing the flow from the second region to the second outlet, e,g, via the valve).

In some embodiments, step ii. results in the retention of at least 90%, at least 95%, at least 97%, at least 99%, between 90 and 99%, between 95 and 99%, or 100% of the initial count of the microorganism in the fluid sample. In some embodiments, step ii. is performed to substantially remove the fluid sample from the first region. In some embodiments, step ii. is performed to substantially transfer the fluid sample into the outlet (e.g. second outlet). In some embodiments, step ii. is performed upon completion of step i. (i.e. upon generating pDEP sufficient for attraction of a bacteria to the first and/or second electrode). In some embodiments, step ii. is completed when the fluid sample is substantially (at least 90%, at least 95%, at least 97%, at least 99%, between 90 and 99%, between 95 and 99%, or 100% of the initial sample volume) transferred into the fluid chamber. In some embodiments, step ii. is completed when the at least 90%, at least 95%, at least 97%, at least 99%, between 90 and 99%, between 95 and 99%, or 100% of the initial sample volume is transferred into the outlet (e.g. the second outlet).

In some embodiments, the method of the invention comprises a washing step comprising introducing a liquid (e.g. the first aqueous solution) into the apparatus while applying AC to the second electrode so as to generate pDEP at the second location. In some embodiments, the washing step is performed without applying AC to the first electrode (or the AC is turned off while performing the washing step). In some embodiments, the washing step is performed upon completion of the step ii. In some embodiments, the washing step is performed prior to extracting the microorganism (step iii.). In some embodiments, the washing step results in or is accompanied by retention of the microorganism at the second location. In some embodiments, the washing step results in a translocation of the microorganism. In some embodiments, the washing step is performed to substantially remove the solid and liquid constituents of the fluid sample, which are not the microorganism. In some embodiments, the washing step is performed to substantially remove the inhibitors from the fluid sample.

In some embodiments, the washing step comprises introducing the first aqueous solution, and inducing a flow of the first aqueous solution within the fluid chamber towards the outlet (e.g. the second outlet). In some embodiments, the washing step comprises introducing at least 0.1, at least 1, at least 2, at least 5, at least 10, or between 1 and 10 fluid volumes of the first aqueous solution.

In some embodiments, the first aqueous solution is characterized by (i) electrical conductivity of at least 0.03 mS/cm, at least 0.05 mS/cm, at least 0.1 mS/cm, or between 5 and 1000 mS/m, between 5 and 500 mS/m, between 5 and 100 mS/m, between 5 and 200 mS/m, between 1 and 1000 mS/m, between 10 and 1000 mS/m, between 50 and 1000 mS/m, between 100 and 1000 mS/m, between 100 and 1000 mS/m, between 200 and 1000 mS/m, including any range between; by (ii) viscosity at 25° C. between 1 and 1000 cP, between 1 and 10 cP, between 1 and 100 cP, between 1 and 500 cP, between 1 and 200 cP, including any range between, or both (i) and (ii). In some embodiments, the first aqueous solution has a viscosity between 1 and 10, or less, and is characterized by an electrical conductivity between 0.05 and 2 mS/cm.

In some embodiments, the washing step comprises generating flow (of the first aqueous solution) at a flow rate between about 1 and about 50 μl/min, between about 5 and about 20 μl/min, between about 2 and about 10 μl/min, between about 10 and about 30 μl/min, between about 5 and about 30 μl/min, between about 2 and about 30 μl/min, including any range between.

In some embodiments, step iii. (extraction) comprises introducing a predefined volume of the second aqueous solution into the apparatus, and generating a flow the second aqueous solution within the fluid chamber to extract the microorganism from the fluid chamber into the outlet, thereby obtaining the solution. In some embodiments, step iii. is performed without applying AC to the second electrode and to the first electrode. In some embodiments, step iii. comprises transferring the second aqueous solution from the inlet into the fluid chamber, and inducing a flow of second aqueous solution within the fluid chamber towards the outlet. In some embodiments, step iii. comprises inducing a flow of second aqueous solution from the second region to the first outlet via the first channel.

In some embodiments, step iii. induces detachment of the microorganisms from the second location, and induces a flow of the microorganisms from the second region towards and into the outlet (e.g. in the first outlet). In some embodiments, the predetermined volume of the second aqueous solution is at least 10 times, at least 100 times, at least 1000 times less than the volume of the fluid sample. In some embodiments, the predetermined volume of the second aqueous solution is between 1 and 10 ul. In some embodiments, the flow rate is as described herein.

In some embodiments, the second aqueous solution is water or a salt solution. In some embodiments, the second aqueous solution is a buffer. In some embodiments, the second aqueous solution is a bacterial medium solution. In some embodiments, the second aqueous solution is or comprises a PCR buffer. In some embodiments, the second aqueous solution is or comprises a lysis buffer. In some embodiments, the second aqueous solution is characterized by viscosity at 25° C. of between 1 and 1000 cP, between 1 and 10 cP, between 1 and 100 cP, between 1 and 500 cP, between 1 and 200 cP, including any range between.

In some embodiments, the completion of the steps ii and iii, and of the washing step can be calculated based on the induced flow rate and based on the liquid volume introduced into the apparatus, such as by calculating the time period required for transferring the fluid form the inlet to the outlet.

In some embodiments, the method of the invention comprises performing a preliminary step v. of pre-processing a liquid sample to obtain the fluid sample being substantially devoid of particles e with a particle size above 10 μm, or above 15 μm. In some embodiments, pre-processing comprises filtration. In some embodiments, the preliminary step v. is performed prior to step ii. (sample injection step), or prior to performing step i.

In some embodiments, the preliminary step v. comprises exposing a liquid sample to conditions suitable for substantially removing particles with a particle size above 10 or above 15 μm, thereby obtaining the fluid sample disclosed herein (being substantially devoid of particles with a particle size above 10 or above 15 μm).

In some embodiments, the liquid sample is free-flowable (e.g. is in a liquid state, or in a semi-liquid state). In some embodiments, the liquid sample is a food sample, a water sample, or a biological sample. In some embodiments, the liquid sample is an unprocessed (unfiltered) food sample, a water sample, or a biological sample. In some embodiments, the liquid sample comprises particles with a particle size above 10 or above 15 μm. In some embodiments, the concentration of the particles (having a particle size above 10, or above 15 μm, or more) is above 0.5% w/w, above 1% w/w, above 5% w/w, above 10% w/w, including any range between.

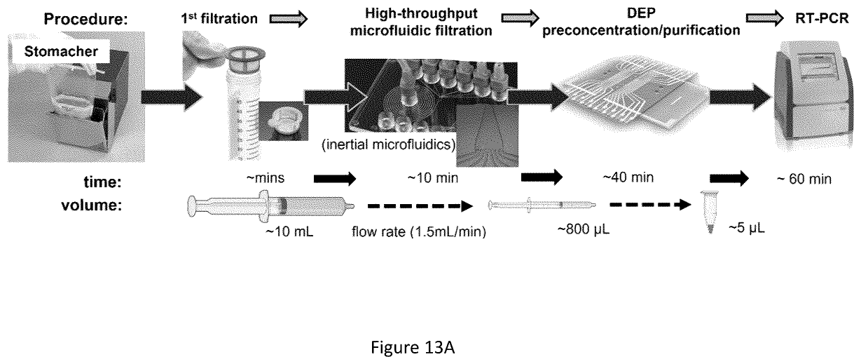

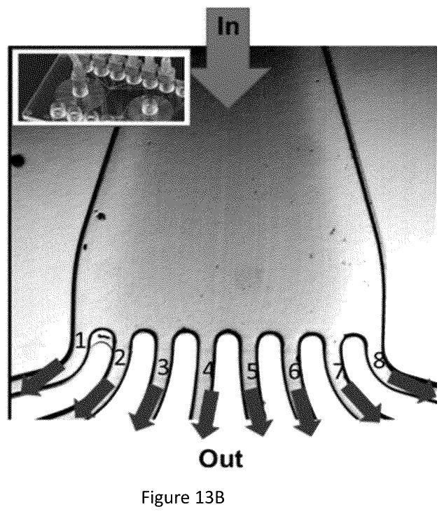

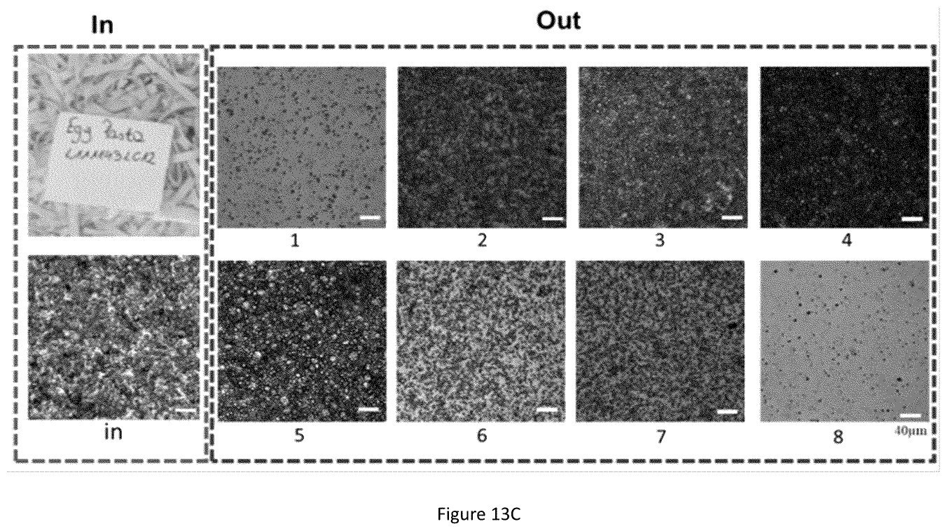

In some embodiments, step v. comprises applying to the liquid sample means suitable for substantially removing the particles therefrom, wherein the means are substantially devoid of membrane filtration. In some embodiments, step v. comprises contacting the liquid sample with a microfluidic sorter (e.g. a microfluidic passive sorter, such as depicted in FIG. 13).

In some embodiments, the method of the invention comprises performing a preliminary step vi. of subjecting a solid sample (e.g. a food sample) to conditions suitable for obtaining the liquid sample (i.e. the flowable sample disclosed hereinabove). In some embodiments, the preliminary step vi. is performed prior to performing the preliminary step v. In some embodiments, the preliminary step vi. comprises diluting a liquid sample with a sufficient amount of an aqueous salt solution (e.g. buffer), to obtain a liquid sample characterized by a conductivity between 0.05 and 2 mS/cm.

In some embodiments, the preliminary step vi. comprises any means suitable for processing a food sample (e.g. a solid or a semi-solid food sample) to obtain the liquid sample. Various methods for the processing of a food sample are well-known in the art, including inter alia dilution (e.g. dilution of a beverage with an aqueous buffer to adjust the conductivity of the sample), cutting, grinding, crushing, blending, enzymatic pre-treatment, including any combination thereof. In some embodiments, the solid food sample is processed by introducing thereof into a blender (e.g. Stomacher) to obtain the liquid sample.

In some embodiments, the preliminary step vi. further exposing the liquid sample to conditions suitable for substantially removing particles with a particle size greater than 40 um, or greater than 80 um. In some embodiments, the preliminary step vi. further comprises exposing the liquid sample to centrifugation, sedimentation, or membrane filtration to remove at least 90-99% of the particles with a particle size greater than 40 um, or greater than 80 um. In some embodiments, the preliminary step vi. comprises filtration of the liquid sample via a membrane with a cut-off of about 40 um, or about 80 um.

In some embodiments, the method of the invention comprises a detection step iv. comprising subjecting the solution to an analytical method suitable for detecting the presence of the microorganism within the solution. In some embodiments, the presence of the microorganism within the solution is indicative of food contamination with the microorganism (e.g. a pathogen). In some embodiments, the step iv. comprises a quantitative detection of the microorganism, allowing deduction of the concentration of the microorganism within the food from which the fluid sample is derived. In some embodiments, the step iv. comprises a sequence specific detection of microorganisms. In some embodiments, the step iv. comprises detection of a microorganism of interest (e.g. one or more specific pathogens).

In some embodiments, the step iv. comprises detecting the presence of one or more DNA or RNA sequence(s) of interest. In some embodiments, detecting is by performing a real-time PCR analysis, or via DNA or RNA sequencing.

In some embodiments, the step iv. comprises DNA and/or RNA detection within the solution, wherein the presence of DNA and/or RNA within the solution is indicative of contamination of the food by the microorganism. In some embodiments, DNA and/or RNA detection (e.g. non-sequence specific detection) is performed via UV spectrometry (e.g. by implementing a NanoDrop Microvolume Spectrophotometer).

In some embodiments, the method of the invention is for detecting a microorganism within a sample (e.g. a liquid or solid food sample/water sample/biological sample, or a sample derived therefrom). In some embodiments, the method of the invention is for detecting a microorganism, wherein the lowest detection limit is between 1 and 100 CFU/ml, between 1 and 1000 CFU/ml, between 1 and 10 CFU/ml, between 10 and 100 CFU/ml, between 100 and 1000 CFU/ml, including any range between.

In some embodiments, the method of the invention is characterized by a lowest detectable amount of DNA/RNA (e.g. bacterial DNA) within a food sample of at least 0.01 pg, at least 0.02 pg, at least 0.03 pg, at least 0.04 pg, including any range between.

In some embodiments, the method of the invention is characterized by a lowest detectable total count of the microorganism within the food sample of at least 1 CFU, at least 5 CFU, at least 2 CFU, at least 10 CFU, including any range between.

In some embodiments, the method of the invention (i.e. at least the steps i., ii, iii., and preliminary steps v.-vi.) is performed under suitable conditions comprising a temperature between about 10 and about 50° C., between about 10 and about 40° C., between about 15 and about 50° C., between about 15 and about 40° C., including any range between.

Reference is now made to FIG. 2 representing a block diagram of an exemplary non-limiting method of the invention. In step 610 (corresponding to the preliminary step vi.), the solid food sample is processed (e.g. in a stomacher) to obtain a liquid sample. In step 620, the liquid sample is filtrated to remove coarse particulate matter, via a filtration membrane with a cut-off of about 40, or about 80 um. In step 630 (corresponding to the preliminary step v.), the liquid sample is introduced into a microfluidic passive sorter, to substantially remove the particles with a particles size above 10 or above 15 um, thereby obtaining the fluid sample. In step 640 (corresponding to the step i.), the fluid sample is introduced into the apparatus of the invention, while applying AC to the first and to the second electrode. In step 640, the microorganism is trapped at the first location and optionally at the second location. In step 650, the washing step is performed (with 1-10 fluid volumes of the first aqueous solution) while applying AC to the second electrode, without applying AC to the first electrode. In step 650, the microorganism is translocated from the first location into the second location, and the inhibitors are substantially removed from the fluid chamber. In step 660, (corresponding to the step iii.) the microorganism is extracted with the second aqueous solution. In step 660, the microorganism is removed the second location into the outlet, thereby obtaining a solution (with a volume of 1-10 ul) consisting essentially of an aqueous carrier and the microorganism. In step 670, the solution is subjected to a method suitable for the DNA/RNA detection (e.g. sequencing or real-time PCR for a sequence specific detection). Step 670 provides indication whether the analyzed food sample is contaminated with a microorganism(s), such as bacterial pathogens.

In the case of a liquid food sample, the step 610 is not performed, and the method starts with step 620.

Optionally, the computer program product comprises a computer-readable storage medium. The computer-readable storage medium may have program code embodied therewith. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list f more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to drawings and/or diagrams of methods, apparatus (systems), and computer program products according to embodiments of the invention. It will be understood that each illustration and/or drawing, and combinations thereof, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the drawings. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the drawings.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the drawings.

In some embodiments, the program code is excusable by a hardware processor.

In some embodiments, the hardware processor is a part of the control unit.

In some embodiments, there is further provided a read-out of the assay carried out in the disclosed system or device may be detected or measured using any suitable detection or measuring means known in the art. The detection means may vary depending on the nature of the read-out of the assay. For example, for assays providing a fluorescent read-out, the detection means may include a source of fluorescent light at an appropriate wavelength to excite the fluorophores in the reaction sites and means detect the emitted fluorescent light at the appropriate wavelength. The excitation light may be filtered using a bandwidth filter before the light is collimated through a lens. The same (e.g., Fresnel) lens may be used for focusing the illumination and collection of the fluorescence light. Another lens may be used to focus the fluorescent light onto the detector surface (e.g., a photomultiplier-tube). Fluorescent read-outs may also be detected using a standard fluorescent microscope fitted with a CCD camera and software. In some embodiments, disclosed system also relates to an apparatus including the device in any embodiments thereof, and a detection means as described herein.

### General

As used herein the term “about” refers to ±10%. Any numerical range or value disclosed herein is considered as being preceded by the term about.

The terms “comprises”, “comprising”, “includes”, “including”, “having” and their conjugates mean “including but not limited to”. The term “consisting of means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

The terms “food”, “food product”, “foodstuff” and “food source” as used herein are interchangeable and refer to any harvested solid, semi-solid or liquid that is suitable for ingestion by a human or domesticated animal and provides nutrition to the human or animal. Examples of suitable harvested solid foods include, without limitation, meat, bread, fish, vegetables, fruit, wet and dry pet foods or treats, and the like. Examples of semi-solid foods include, without limitation, jams, jellies, apple sauce, ice cream, gelatins, puddings, mayonnaise, ketchup, and the like. Examples of suitable liquid foods include, without limitation, water, milk, juices such as fruit or vegetable juices, milk creams, non-dairy liquids such as soy milk, and the like.

The term “pathogenic” refers to disease related conditions including without limitation the ability to render a consumer (i.e., human or domesticated animal) sick or dead.

The term “bacteria” refers to any bacteria whether Gram-positive or Gram-negative and whether aerobic or anaerobic.

The term “pathogenic bacteria” refers to a bacterium which, when ingested by a consumer, is capable of rendering that consumer sick or, in the extreme case, dead.

Examples of pathogenic bacteria include, by way of example only, Listeria, enterohemorrhagic E. coli, Salmonella, botulinum, shigella, and the like.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Example 1

### Dielectrophoretic Bacterial Concentration

Reference is made to FIGS. 1 and 2 providing a non-limiting example of a dielectrophoretic bacterial concentration chip.

The chip may have an inlet reservoir suitable to receive a sample (e.g., a food product), such as a volume of at least 50 μL, at least 100 μL or more. The diameter of the inlet reservoir suitable may be a wide channel suitable for large volume introductions, such as at least 5 mm. The main channel may include a sorting channel having a narrow width, such as of about 0.5 mm and height of about 0.025 mm.

The chip may include an electrode array suitable for negative dielectrophoretic (nDEP) focusing, such as by having a V-shape inclined electrode array. The chip may include an interdigitated electrode array configured to and suitable to apply on-flow electroporation (electrolysis).

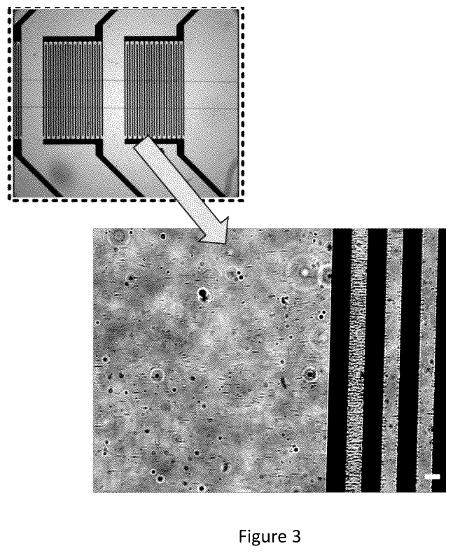

The chip provided herein is able to sort bacteria either using negative dielectrophoretic (nDEP) behavior or positive dielectrophoretic (pDEP) behavior as shown in FIG. 3 and FIG. 8.

As an exemplary, non-limiting goal, attempt was made to detect the pathogenic Gram-positive Listeria monocytogenes and Gram-negative Salmonella. Accordingly, the system was first calibrated using the non-pathogenic strains Listeria innocua ATCC 33090 (Gram-positive) and E. coli ATCC 8739 (Gram-negative) as closely related strains representing the two pathogens. First, the conditions for the efficient concentration of the two strains were established.

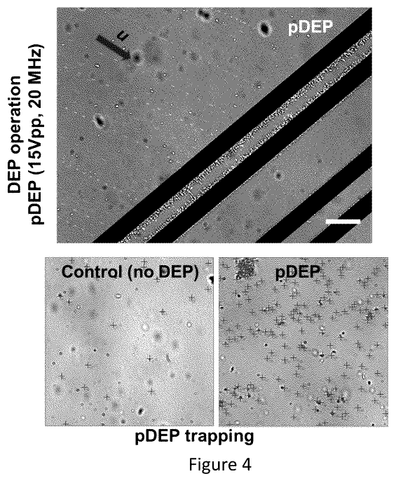

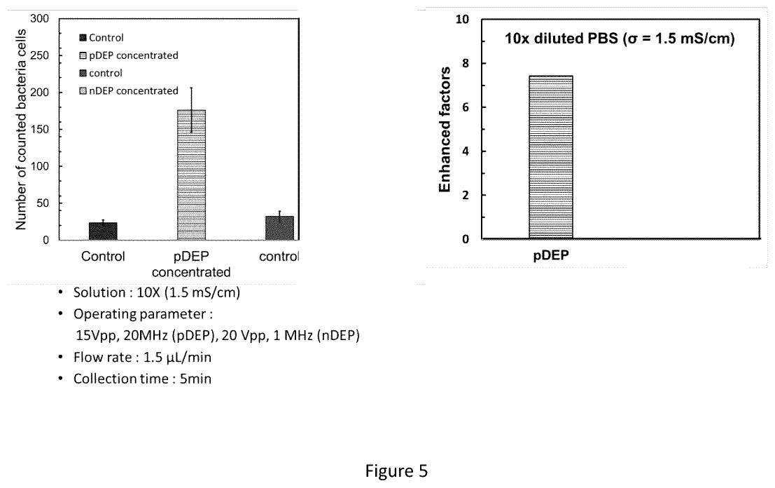

FIG. 4 depicts the microscope images of either pDEP or nDEP trapping/sorting and the difference between the control (i.e. without preconcentration) versus the preconcentrated case. FIG. 5 depicts the respective quantitative trapping efficiency of either nDEP or pDEP versus the non-concentrated (i.e. control) case exhibiting an enhancement factor of ˜8.

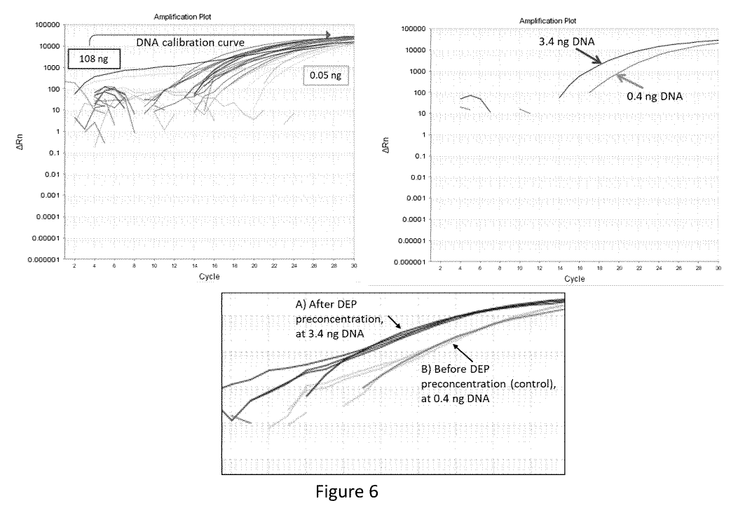

Using specific DNA primers for E. coli, Real-Time PCR was conducted and compared the difference in Ct (cycle threshold) between control cells (before concentration) and cells subjected to DEP (after concentration). A clear enhancement in the Ct values owing to concentration of the cells was observed using the device and method provided herein (FIG. 6). The level of Real-Time PCR detection enhancement when comparing control non-concentrated cells to nDEP-concentrated cells was in the order of ×10 based on our DNA calibration curve (FIG. 6).

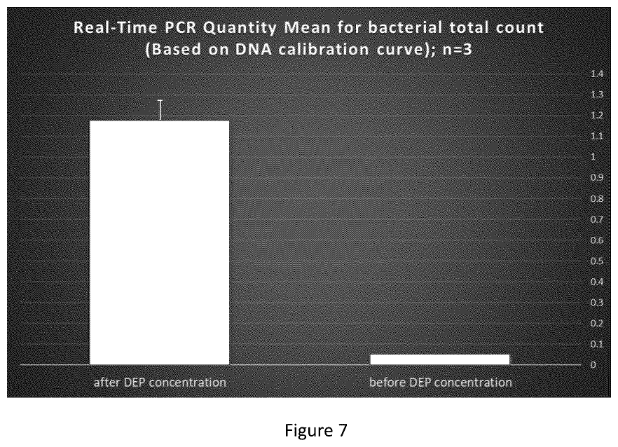

Using specific DNA primers for bacterial total count, both Gram-positive and Gram-negative (based on Genes Coding for Bacterial 16S rRNA), Real-Time PCR was conducted and compared the difference in Ct (cycle threshold) between control cells (before concentration) and cells subjected to DEP (after concentration). A clear enhancement in the Ct values owing to concentration of the cells was observed using the device and method provided herein as demonstrated by a significantly higher Quantity Mean value (FIG. 7). The level of Real-Time PCR detection enhancement when comparing control non-concentrated cells to nDEP-concentrated cells was at least in the order of ×30 based on the relevant DNA calibration curve (FIG. 7). This result is especially significant for detection of total bacteria at low concentrations, a few hundred cells, as indicated by such tests we perform at the lower end of the Real-Time PCR limit of detection,

The chip provided herein is able to preconcentrate either specific bacteria or all bacterial cells using positive (pDEP) and/or negative (nDEP) dielectrophoretic behavior using an electrode array embedded on either the bottom or on both top and bottom walls of the channel as shown in FIG. 8.

Extended to [055], the chip provided herein composes multi-stacked DEP electrode arrays and multi-stacked channels within common inlets for high-throughput concentration of bacterial cells as shown in FIG. 8b.

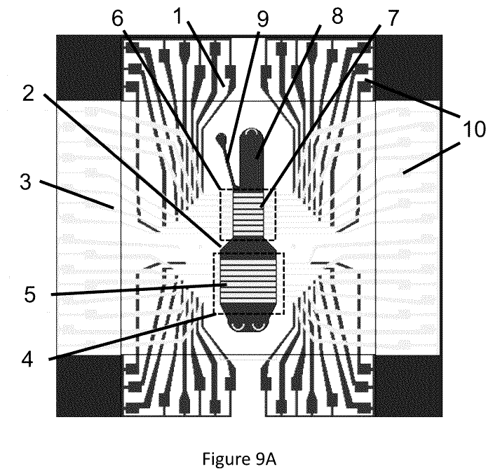

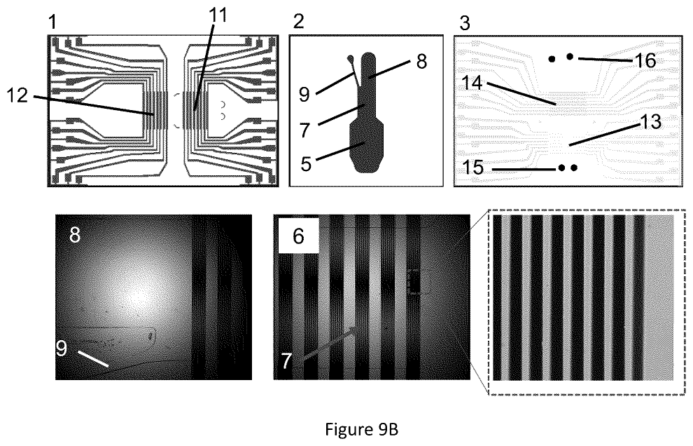

FIG. 9 depicts a single-stacked three-dimensional DEP electrode array chips which enables to introduce a large volume of at least 300 μL or more with flow rate around 10 μL/min. The chip consists of multiple DEP-electrode arrays which produce the same or different functionality (e.g., 1^{st} electrode array: efficient trapping, 2^{nd} electrode array: volume reduction for efficient sorting). As a representative design, an pDEP electrode array with interdigitated structure was used for both 1^{st} and 2^{nd} DEP electrode array.

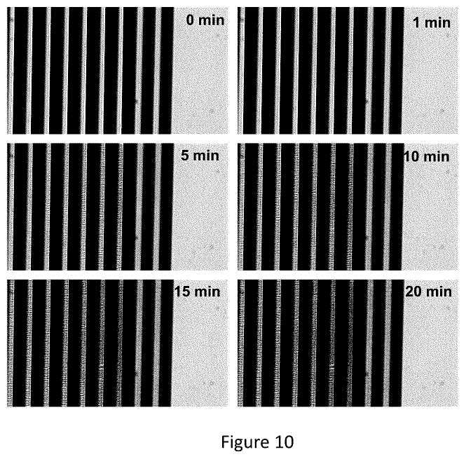

FIG. 10 depicts the time-lapse microscope images showing pDEP trapping of bacterial cells (Listeria innocua ATCC 33090) for 30 min with ˜350 μL introduction.

In the food industry, when testing for Salmonella and Listeria as the leading pathogens of interest, most tested samples are at very low bacterial concertation. Therefore, they are below the threshold for identification prior to enrichment. The concentration device and method provided herein is of high potential to revolutionize the testing market as it will decrease the duration of enrichment step needed for detection of those important pathogens, allowing for more sensitive, at least by an order of magnitude, more robust and quick diagnosis.

### Example 2

### Positive DEP Enhanced Bacterial Detection

Based on solid experimental results (not shown), the inventors surprisingly found that the induction of positive DEP (pDEP) within the apparatus of the invention is essential for an efficient entrapment of microorganisms (e.g. bacterial cells) within the first and/or second location within the fluid chamber, as disclosed hereinabove.

The following results show enhanced Real-Time PCR detection owing to DEP of a representative pathogen (Listeria), of total count where both Gram-positive and Gram negative bacteria are detected, and with food types that are challenging to test in terms of food texture and/or its inhibitory negative impact on PCR. All tests were performed directly on the samples, without the time-consuming and costly step of DNA extraction.

### Listeria Detection

As the first step of optimization for microorganism detection (e.g. detection of bacterial cells, in particular the Gram-positive Listeria) using the apparatus and/or method disclosed herein, the inventors calibrated the molecular biology setup in terms of both the bacterial DNA calibration and the range of CFU/mL concentration detectable by real-time PCR (FIG. 2). the inventors postulated that for efficient trapping by DEP, the food sample (or the fluid sample disclosed herein) should be diluted (at least 2 times, at least 5 times at least 10 times, or between 2 and 100 time, between 2 and 50 times, between 2 and 20 times dilution).

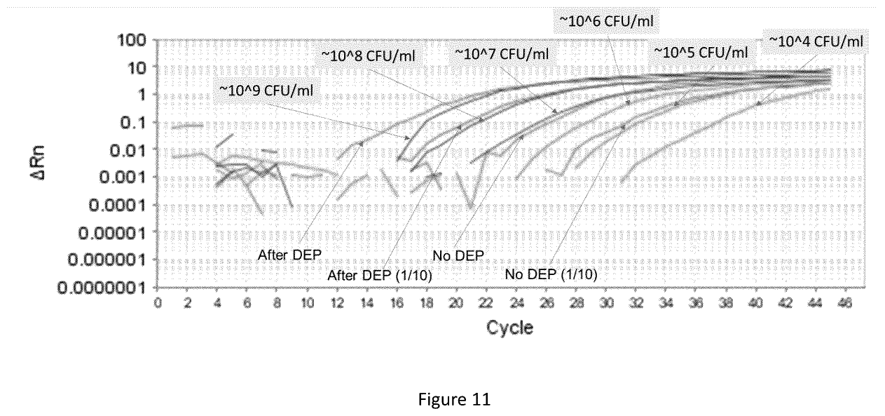

PCR primers for specific amplification of Listeria innocua were used for detecting the bacteria. In terms of DNA calibration, the inventors were able to detect as little as 0.04 pg pf Listeria genomic DNA. This was demonstrated in parallel as ˜10\hat{ }4 CFU/mL or as few as about 10 CFU only, detected by real-time PCR. The processing of such Listeria-contaminated apple juice via DEP on chip, was achieved at nearly 100 times enhancement of detection (FIG. 11) when comparing DEP versus no-DEP following a 10× dilution of the enrichment buffer for effective DEP trapping. When comparing the DEP versus the no-diluted the enhancement factor is about an order of magnitude.

Using the method and the apparatus of the invention, the inventors significantly enhanced sensitivity of real-time PCR (around two orders of preconcentrating factor relative to the diluted sample and around one order of magnitude enhanced sensitivity relative to the non-diluted sample) with different microorganisms (e.g. pathogenic bacteria) and at lowest detection limit of as little as a few dozens of CFUs only. This is important both in terms of detection of minimum levels of contamination, which often go undetected and cause later disease and recalls, as well as the ability to decrease the duration of the required enrichment time of samples suspected to contain pathogenic bacteria. It is important to note that the method of the invention results in a significant time and costs saving, since the preconcentrated bacterial sample (also referred to herein as “solution”) is directly PCR amplified without any DNA purification step.

### Total Count Detection

Total count detection is a highly common test in the food industry and in fact all commercial products and materials that may be of contamination risk, are first tested for total count, or the presence and number of bacteria. This important screening process is often used to verify absence of contamination even at the lowest levels that may not be detected by PCR. Total count is performed for both Gram-positive and Gram-negative bacteria and usually takes one to two days. The method and the apparatus disclosed allows for a detection of a variety of microorganisms (e.g. plurality of different bacterial species), all in one test. This was confirmed for instance, for the Gram-positive Listeria and the Gram-negative E. coli in different tests yet with the same PCR primers, demonstrating the wide range of detection capabilities of the primers.

### Example 3

pDEP-Based Purification of Bacteria and Removal of PCR Inhibitors in the Food Samples

When testing food samples for total count, they were tested directly, without an enrichment step. An important observation the inventors noticed during validation tests on different types of food, was the inhibitory impact of some foods on PCR. Certain food types were not detectable by real-time PCR, due to inhibitory components in the food, preventing the reaction from occurring. DEP enables not only to preconcentrate bacteria cells by entrapping them onto the DEP electrodes, but also to sort most food components that are inhibitory to PCR.

After food introduction with pDEP-based preconcentration, the solution was replaced to a diluted BH buffer (σ=0.5 mS/cm) for a purification step, which was followed by a collection step. During the purification step, all trapped bacteria cells on the DEP electrode array were transported downstream and re-trapped at the last DEP electrode array located at the end of a downstream channel, by selective DEP operation for efficient bacteria collection.

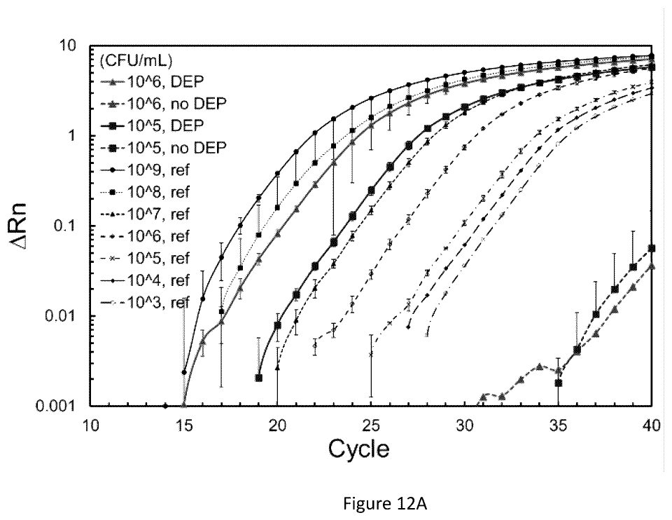

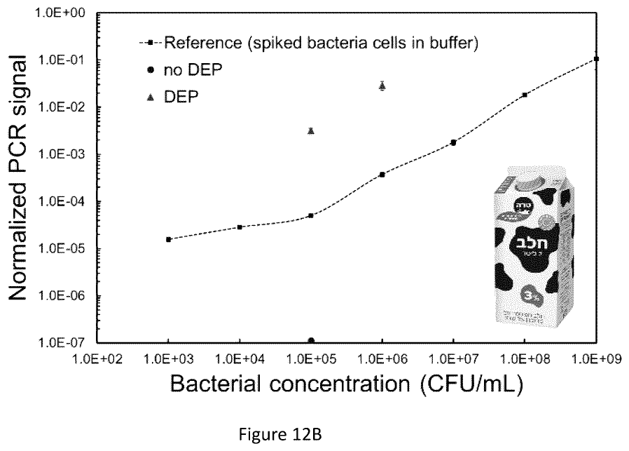

FIG. 12 clearly demonstrates the DEP-based enhancement of bacterial detection in a PCR-inhibitory product such as milk. The enhanced detection obtained is thus a result of two impacts combined-both the preconcentration of the bacteria and the washing away of the inhibitors. The preconcentration factor is determined by volume reduction (i.e., volume of introduced milk solution (˜700 μL) divided by the volume of bacterial cells within a purified buffer (˜5 μL) which is also well-matched by comparing the results of ‘DEP’ (both 10\hat{ }5 and 10\hat{ }6 CFU/mL in milk) and reference (10\hat{ }7 and 10\hat{ }8 CFU/mL without milk). The effect of only the latter can be appreciated by comparing the reference of the 10\hat{ }5 and 10\hat{ }6 CFU/mL versus those obtained in the ‘No DEP’ (10\hat{ }5 and 10\hat{ }6 CFU/mL and spiked in milk) where the latter two results within milk are completely inhibited. It should be emphasized that the most common tests of bacterial contamination in milk require an expensive step of DNA extraction, which is avoided when using the method disclosed herein.

### Example 4

### Pre-Filtering of Food Sample to Prevent Clogging of the Fluid Chamber

Some food types are challenging for detection of contamination as they may be too coarse (chunky) and difficult to process. For those food types, we combined two microfluidic technologies which are an inertial microfluidic based high-throughput filtration and DEP-based bacterial preconcentration (FIG. 13A). As an example of a test where pre-filtering of products is required, we took egg pasta, which was inoculated with E. coli (FIGS. 4b and c). After processing the sample using a 250 μm filter bag processed (of note, this step is optional and can be skipped) via stomacher for 2 minutes, the sample was filtered again using a 40 μm Cell Strainer. We next used a commercially available microfluidic chip (inertial microfluidic chip, Chipshop) for sorting bacterial cells from the large-sized sample components of 10 to 40 μm in diameter. This step prevents the unwanted clogging event during DEP trapping in a 25 μm channel height (FIG. 13). The optimized flow rate for sorting is 1.5 mL/min. We load 10 mL of food samples including bacteria cells and obtain ˜1 mL of the sorted food solution from outlet 8 (FIGS. 13b and 13c), which is relatively clean from large-sized particles, while maintaining the same initial bacterial concentration.

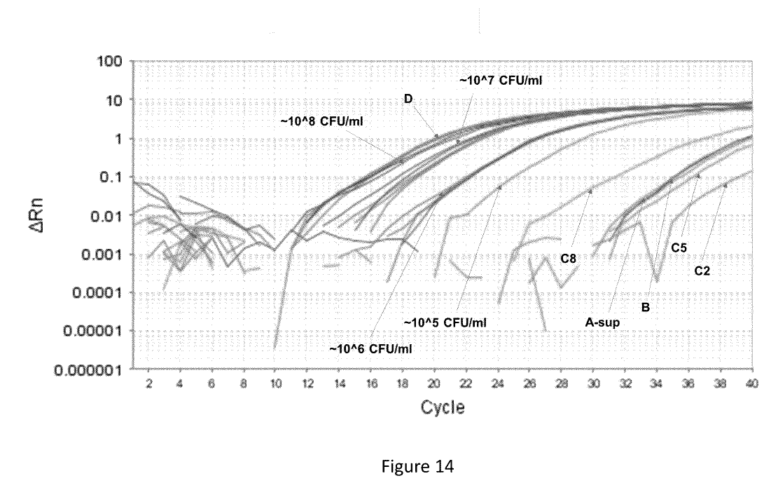

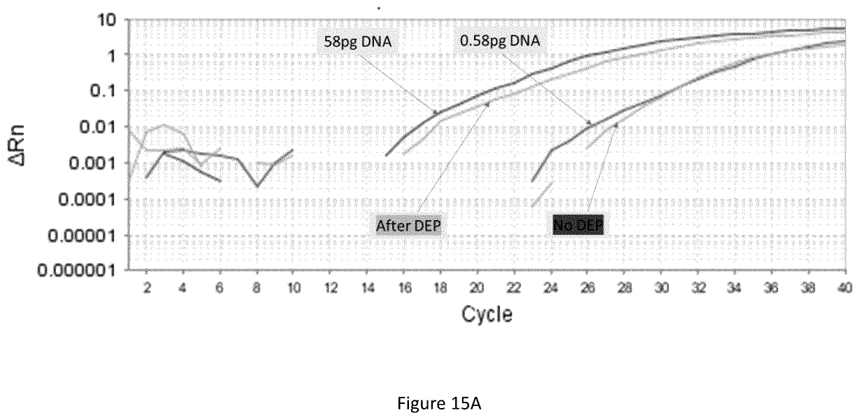

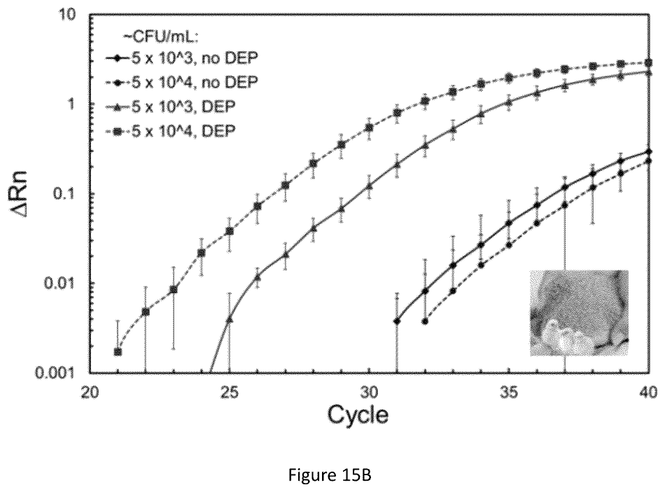

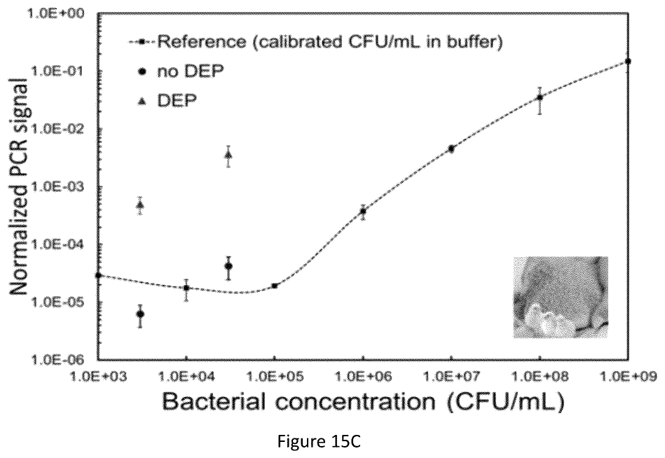

FIG. 14 shows the resulting PCR signal from the bacteria cells in egg pasta from various outputs of an exemplary method of the invention as illustrated in FIG. 13a (i.e, supernatant from stomacher (‘A-sup’), after 40 μm cell strainer (‘B’), outputs from inertial microfluidic chip (‘C’) and after DEP (‘D’) where 8*10\hat{ }6 CFU/mL of E. coli cells were spiked into the food. During the DEP operation, we introduced 250 μL of the pre-filtered sample solution (inertial outlet 8, ‘C8’) and collected ˜5 μL after preconcentration/purification. By comparing PCR results of C8 and B or A-sup, we can see the improvement of signal at C8, after DEP: a significant enhancement in detection of total count. The main role of the inertial microfluidic technology is a rapid filtration of large contaminants which cause clogging of the DEP chip, while substantially maintaining the initial concentration of bacterial cells in the fluid sample. Further, pre-filtering was tested for a number of challenging food types. FIG. 15 demonstrates how owing to the combination of pre-filtering and DEP, the enhancement in signal was over about two orders of magnitude. As demonstrated in FIG. 15A, the inventors successfully detected as low as about 0.6 pg of DNA within a food sample. As demonstrated in FIG. 15B, the inventors successfully detected the presence of bacteria in a food sample even at a low concentration of about 10\hat{ }3 CFU/ml.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. An apparatus comprising:
a fluid chamber comprising a wall shaped and sized for receiving a fluid sample suspected of containing a microorganism;
said fluid chamber is characterized by a minimal width of about 0.6 mm and by a height between about 10 and about 50 μm;
comprises a first region and a second region downstream and in fluid communication with the first region, wherein a cross-section of the first region is greater than a cross-section of the second region;
a first electrode, electrostatically coupled to said first region, and configured to generate a positive dielectrophoresis (pDEP) force at a first location within the first region;
a second electrode, electrostatically coupled to said second region, and configured to generate a pDEP force at a second location within the second region.

2. The apparatus of claim 1, wherein each of said first electrode and said second electrode is in operable communication with an electric source configured to apply alternating current (AC); wherein said fluid chamber is configured to support a flow of said fluid sample, wherein the flow is between about 10 and about 50 μl/min; wherein said fluid chamber further comprises an inlet suitable for introducing the fluid sample, and optionally an outlet; and wherein the inlet and the outlet are in fluid communication with fluid chamber.

3. (canceled)

4. The apparatus of claim 1, wherein said microorganism is selected from at least one bacterial cell, at least one yeast cell, and at least one spore.

5. (canceled)

6. The apparatus of claim 1, wherein said fluid chamber is characterized by at least one of: a minimal length of about 10 mm, and by a width between about 0.6 and about 2 mm wherein said wall is characterized by a thickness of at least 10 μm; and wherein said wall consists essentially of a dielectric material.

7. The apparatus of claim 1, wherein said first electrode is in a form of a first array of electrodes; and said second electrode is in a form of a second array of electrodes; and wherein said first array and said second array are positioned on top of the wall, or embedded within the wall.

8. The apparatus of claim 1, wherein a ratio between the cross-section of the first region to the cross-section of the second region within the fluid chamber is between about 1:2 and 1:3.

9. (canceled)

10. A method for detecting a microorganism within a fluid sample, the method comprising:
i. applying AC to said first electrode and to said second electrode of the apparatus of claim 1, to generate pDEP;
ii. introducing the fluid sample into the apparatus while inducing a flow of said fluid sample within the fluid chamber, wherein in response to pDEP the microorganism is retained (i) at the first location, (ii) at the second location, or both (i) and (ii), thereby inducing a flow of the fluid sample within the fluid chamber;
iii. extracting said microorganism from the fluid chamber by introducing a predefined volume of an aqueous solution into the apparatus, thereby obtaining a solution; and
iv. subjecting said solution to an analytical method suitable for detecting the presence of the microorganism within the solution; wherein said fluid sample is suspected of containing the microorganism and is substantially devoid of a particulate matter with a particle size above 15 μm; and wherein a volume of said solution is at least 10 times lower than volume of the fluid sample.

11. The method of claim 10, wherein the flow of said fluid sample within the fluid chamber is between 10 and about 50 μl/min.

12. The method of claim 10, wherein the predefined volume of the aqueous solution is between 1 and 10 ul.

13. The method of claim 10, wherein the step iii. is performed without applying AC to said first electrode and to said second electrode; and wherein said method is performed under suitable conditions comprising a temperature between about 10 and about 50° C.

14. (canceled)

15. The method of claim 10, wherein said fluid sample is characterized by a viscosity of the between about 1 and 100 cP; and by electrical conductivity between 0.05 and 2 mS/cm; and wherein a volume of said fluid sample is up to about 2 ml.

16. (canceled)

17. The method of claim 10, wherein the fluid sample comprises a food sample, a water sample, or is derived (i) from a food sample, (ii) from a water sample, or (iii) from a biological sample; and wherein the method further comprises performing a washing step prior to performing the step iii, wherein the washing step comprises introducing at least 1 fluid chamber volume of an aqueous solution into the apparatus and inducing a flow of said aqueous solution within the fluid chamber, while applying AC to the second electrode, so as to generate a pDEP at the second location.

18. (canceled)

19. The method of claim 17, wherein the washing step comprises any one of: (i) introducing about 10 fluid chamber volumes of the aqueous solution; and (ii) wherein said flow is between about 5 and 30 ul/min; and wherein in response to the pDEP the microorganism is retained at the second location.

20. (canceled)

21. The method of claim 10, optionally comprising a preliminary step v. of subjecting a liquid sample comprising particles with a particle size above 10 μm to conditions suitable for substantially removing said particles, thereby obtaining the fluid sample; wherein said preliminary step v. is performed prior to performing said step ii.

22. The method of claim 21, wherein said conditions comprises contacting the liquid sample with a microfluidic sorter; wherein the method further comprises a preliminary step vi. of subjecting a food sample to conditions suitable for obtaining the liquid sample of claim 21; wherein said preliminary step vi. is performed prior to performing the preliminary step v; and wherein said conditions comprise subjecting said food sample to a filtration to substantially remove particles with a particle size of at least 40 um.

23. (canceled)

24. (canceled)

25. The method of claim 10, wherein a lowest concentration of said microorganism within the fluid sample detectable by said method is between 1 and 100 CFU/ml.

26. The method of claim 10, wherein the analytical method comprises detecting the presence of a DNA or RNA sequence of interest wherein the DNA or RNA sequence of interest is derived from said microorganism, optionally wherein the microorganism is a pathogen; optionally wherein detecting is by performing a real-time PCR analysis, or via DNA or RNA sequencing.

27. (canceled)

28. (canceled)
